(19) United States
(12) Patent Application Publication
(10) Pub. No.: US 2011/0118202 A1
(43) Pub. Date: May 19, 2011

(54) Molecules which Bind to the Dimerization Initiation Site (DIS) of HIV RNA, Their Synthesis and Their Applications as Drugs

(76) Inventors: Patrick Pale, Strasbourg (FR); Jean-Marc Weibel, Huttentheim (FR); Anne Bodlenner, Graz (AT); Roland Marquet, Illkirch-graffenstaden (FR); Jean-Cristophe Paillart, Strasbourg (DE); Philippe Dumas, Strasbourg (FR); Eric Ennifar, Strasbourg (DE); Philippe Walter, Strasbourg (DE)

(21) Appl. No.: 12/225,356

(22) PCT Filed: Mar. 23, 2007
(86) PCT No.: PCT/IB2007/002125

§ 371 Date: Jan. 31, 2011

Related U.S. Application Data
(60) Provisional application No. 60/785,517, filed on Mar. 23, 2006.

(51) Int. Cl. A61K 31/7036; C07H 15/232; C07H 15/224; C07H 15/23; A61P 31/18

(52) U.S. Cl. 514/39; 514/36; 514/38; 536/13.2; 536/16.6; 536/16.8

(57) ABSTRACT

The dimerization of HIV RNA is a key step in the virus replication cycle. Based on RNA DIS crystal structures, a novel kind of compounds, dimeric or not, based on neamine was designed and synthesized. Biological studies showed that such compounds bind and interfere with the targeted RNA sequence, opening a new anti-HIV approach. The crystal structures and bio-chemical experiments showed that the DIS of HIV-1 genomic RNA is a target for new anti-HIV drugs and that those drugs could be derived from aminoglycosides. The results revealed that binding of aminoglycosides to the DIS is specific regarding both the aminoglycoside family and the RNA subtype.

### CROSS-REFERENCE TO RELATED APPLICATION

The present application claims the benefit of U.S. Provisional Application Ser. No. 60/785,517, filed Mar. 23, 2006, which is incorporated herein by reference in its entirety.

### FIELD OF THE INVENTION

The present invention relates to novel anti-Human Immunodeficiency Virus Type 1 (HIV-1) drugs. More particularly, the present invention relates to the synthesis of ligands and aptamers that selectively target the stem loop (SL1) kissing-loop complex formed by the dimerization initiation site (DIS) of HIV-1 ribonucleic acid (RNA). More specifically, the present invention relates to the synthesis and use of aminoglycosides that selectively target the kissing-loop complex by preventing either the dissociation of the kissing-loop complex or its formation, thereby inhibiting replication of the HIV-1 RNA as well as the use of aminoglycosides that target the corresponding extended duplex. The invention further relates to a composition comprising such aminoglycosides and to a method of using these compositions in medical therapeutic and diagnostic procedures.

### BACKGROUND OF THE INVENTION

It is estimated that there are approximately 40 million cases of HIV infection and AIDS worldwide. There are steep rises of new infections in Africa, Eastern Europe and Central Asia. AIDS is the primary cause of death among young men and women in the Caribbean region. In Asia, some 7 million people are living with HIV. In industrialized countries, AIDS continues to have a significant impact in minority communities where complacency in the face of a major health risk is a growing problem (Report of the Executive Director of the Joint United Nations Program on HIV/AIDS (UNAIDS) December, 2006).

HIV-1 is a retrovirus and utilizes RNA as its genomic message. Genome packaging is directed by a gag polypeptide produced in the host cell during late stages of the infectious cycle. An element of gag that is essential for genome recognition and the packaging of infectious RNA is a 55 amino acid nucleocapsid protein, NCp7. As part of gag, NCp7 initiates genomic RNA encapsidation by recognition of a ca. 120 nucleotide sequence of the RNA genome that contains four stem-loop (SL) sequences in its secondary structure (e.g., SL1, SL2, SL3, and SL4). Although multi-drug therapy of AIDS with inhibitors of HIV-1 reverse transcriptase and HIV-1 protease has dramatically delayed the onset of clinical disease and death due to AIDS, problems with this therapy are of increasing concern.

Currently available drugs for the treatment of HIV include six nucleoside reverse transcriptase (RT) inhibitors (zidovudine, didanosine, stavudine, lamivudine, zalcitabine and abacavir), three non-nucleoside reverse transcriptase inhibitors (nevirapine, delavirdine and efavirenz), and five peptidomimetic protease inhibitors (saquinavir, indinavir, ritonavir, nelfinavir and amprenavir). Each of these drugs can only transiently restrain viral replication if used alone. However, when used in combination, these drugs have a profound effect on viremia and disease progression. In fact, significant reductions in death rates among AIDS patients have been recently documented as a consequence of the widespread application of combination therapy. However, despite these impressive results, 30 to 50% of patients ultimately fail combination drug therapies. Insufficient drug potency, non-compliance, restricted tissue penetration and drug-specific limitations within certain cell types (e.g. many nucleoside analogs cannot be phosphorylated in resting cells, which is required for biological activity) may account for the incomplete suppression of sensitive viruses. Furthermore, the high replication rate and rapid turnover of HIV-1 combined with the frequent incorporation of mutations, leads to the appearance of drug-resistant variants and treatment failures when sub-optimal drug concentrations are present. Therefore, novel anti-HIV agents exhibiting distinct resistance patterns, and favorable pharmacokinetic as well as safety profiles are needed to provide more treatment options.

Currently marketed HIV-1 drugs are dominated by either nucleoside reverse transcriptase inhibitors or peptidomimetic protease inhibitors. Non-nucleoside reverse transcriptase inhibitors (NNRTIs) have recently gained an increasingly important role in the therapy of HIV infections. At least 30 different classes of NNRTI have been described in the literature and several NNRTIs have been evaluated in clinical trials. However, the major drawback to the development and application of NNRTIs is the propensity for rapid emergence of drug resistant strains, both in tissue cell culture and in treated individuals, particularly those subject to monotherapy.

It is clear that there is a need for anti-HIV drugs targeted against novel viral targets that are less prone to the development of resistant HIV virus. These facts stress the importance of methods for the identification of new anti-HIV molecules or compounds and HIV targets.

Increasing evidence has suggested that RNA may become a prime target for antiviral therapy (Gallego et al., Acc. Chem. Res. 2001, 34:836-843; Hermann, Chem. Int. Ed. 2000, 39:1890-1905), as supported by a growing interest for small molecules capable of interfering with RNA. Among these small molecules, aminoglycosides are arguably the most studied and best characterized. Aminoglycosides have become leading structures for studying RNA-ligand interactions as well as for designing novel ligands for nucleotides (Tok et al., Curr. Topics Med. Chem. 2003, 3:1001-1019; Seeberger et al., Synlett. 2003, 9:1323-1326). Many aminoglycoside derivatives have shown antiviral activity by binding to specific regions of viral RNA (Luedtke et al., Biochemistry 2003, 39:11391-11403; Arya et al., Bioorg. Med. Chem. Lett. 2004, 14:4643-4646; Liu et al., J. Am. Chem. Soc. 2004, 126:9196-9197; Litovchick et al., Biochemistry 2000, 39:2838-2852).

One of the many features of aminoglycosides resides in their ability to recognize secondary and tertiary structures of RNA in which the base pairing has been disrupted (e.g. bulges, internal loops and stem junctions). A specific example of such secondary and tertiary structures constitutes the bulge regions of unrelated RNA sequences from the 16S ribosome, HIV TAR, HIV RRE, and the Group I intron (Wang et al., J. Am. Chem. Soc. 1997, 119:8734-8735; Arya et al., J. Am. Chem. Soc. 2001, 123:5385-5395). Furthermore, aminoglycosides specifically bind to kissing-loop complexes formed by the RNA dimerization initiation site of the HIV virus (Russell et al., J. Am. Chem. Soc. 2003, 124:3410-3411), stabilize DNA-RNA triplexes and hybrid duplexes, and even induce hybrid triplex formation (Sucheck et al., J. Am. Chem. Soc. 2000, 122:5230-5231; Arya et al., J. Am. Chem. Soc. 2003, 125:10148-10149).

Successful replication of HIV requires an ordered pattern of viral gene expression (Vaishnav et al., Annu. Rev. Biochem. 1991, 60:577-630; Emerman et al., Science 1998, 280:1880-1884), and several essential viral RNA binding proteins. Dimerization of genomic RNA is ubiquitous among retroviruses (Kung et al., Cell 1976, 7:609-620; Paillart et al., Biochimie 1996, 78:639-653). Dimerization is beneficial for reverse transcription, including recombination, and is intricately linked to encapsidation of the genomic RNA and to the morphogenesis of the mature viral core (Paillart et al., Nature Rev. Microbiol. 2004, 2:461-472). In Human Immunodeficiency Virus Type 1 (HIV-1), packaging of HIV-1 RNA as a dimer is essential for an efficient viral replication (Berkhout et al., J. Virol. 1996, 70:6723; Clever et al., J. Virol. 1997, 71:3407-3414; Paillart et al., J. Virol. 1996, 70:8348-8354; Shen et al., J. Virol. 2000, 74:5729-5735). In mature virus particles, the retroviral genome is noncovalently dimeri zed near its 5′-end.

This dimeric nature has been demonstrated to be critically important for several key events in the life cycle of HIV, including reverse transcription and encapsidation (Russell et al., Retrovirology 2004, 1:23-26; Paillart et al., Nat. Rev. Microbiol. 2004, 2:461-472; Berkhout et al., J. Virol. 1996, 70:6723-6723).

The dimerization initiation site (DIS) of the genomic HIV-1 RNA is a stem-loop (SL1) motif characterized by a nine nucleotide-loop. The loop contains a six nucleotide self complementary sequence flanked by purines (FIG. 1). The same self-complementary sequence is found in all HIV subtypes, except in subtypes B and D, and flanking nucleotides are mostly purines. This sequence allows for the formation of a loop-loop ‘kissing complex’ (Skripkin et al., PNAS 1994, 91:4945-49). Mutations in SL1 are characterized by unstable and/or aberrant RNA dimers; they affect RNA packaging and reverse transcription, and result in strongly diminished infectivity (up to 100,000-fold) (Paillart et al., J. Virol. 1996, 70:8348-8354; Berkhourt et al., J. Virol. 1996, 70:6723-6732; Shen et al., J. Viral. 2000, 74:5729-5735; Clever et al., J. Viral. 1997, 71:3407-3414).

High-resolution X-ray structures of the kissing-complex form for subtypes A, B and F, (Ennifar et al., Nat. Struct. Biol. 2001, 8:1064-1068; Ennifar & Dumas, J. Mol. Biol. 2006, 356:771-782.) and of the extended duplex form for subtypes A and B (Ennifar et al., Structure 1999, 7:1439-1449; Ennifar et al. Nucleic Acids Res. 2003, 31:2671-2682) have been determined. The kissing-complex form is mainly characterized by a coaxial interaction of the two stem-loops, the two conserved purines 5′ of the self-complementary sequence being stacked on one another and bulged out (FIG. 2).

Nucleic acid ligands or aptamers are nonencoding single-stranded nucleic acid (DNA or RNA) that have the property of binding specifically to a desired target compound or molecule, and that have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

Nucleic acid ligands possess a number of features that can render them useful as therapeutic agents. They can be made as relatively small (e.g., 8 kDa to 15 kDa) synthetic compounds and can be selected to possess high affinity and specificity for target molecules (equilibrium dissociation constants ranging from, for example, 0.05-10 nM). Aptamers embody both the affinity properties of monoclonal antibodies and single chain antibodies and the manufacturing ease similar to that of a small peptide. Initial studies demonstrated the in vitro use of aptamers for studying protein function, and more recent studies have confirmed the utility of these compounds for studying in vivo protein function (Floege et al., Am. J. Pathol. 1999, 154:169-179; Ostendorf et al., J. Clin. Invest. 1999, 104:913-923). In addition, animal studies to date have shown that aptamers and compounds of similar composition are well tolerated, exhibit low or no immunogenicity, and are thus suitable for repeated administration as therapeutic compounds (Floege et al., Am. J. Pathol. 1999, 154:169-179 ; Ostendorf et al., J. Clin. Invest. 1999, 104:913-923; Griffin et al., Blood 1993, 81:3271-3276; Hicke et al., J. Clin. Invest. 2000, 106:923-928).

Currently, many drugs elicit medical complications such as side effects and undesirable or uncontrollable outcomes. Treating medical complications that result from side effects leads to additional healthcare costs. The recent identification of a range of nucleic acid ligands useful in medical therapy has opened new avenues of research and development. While progress has been made in this area, a strong need remains to provide methods and compositions to improve the manner in which these ligands are used and to increase their efficacy, to better control the process of therapy, and to provide therapies that have decreased side effects over traditional therapeutic methods. The present invention provides compounds, compositions and methods to improve the process of using nucleic acid ligands in medical therapy. The approach provided by the present invention allows for more control over the therapeutic effect, pharmacokinetics and duration of activity of nucleic acid ligands.

What is needed are new anti-HIV molecules and drugs that target key steps in the virus replication cycle, and molecules and drugs that are suitable for repeated administration as therapeutic compounds.

### SUMMARY OF THE INVENTION

The present invention comprises new anti-Human Immunodeficiency Virus Type-1 (HIV-1) drugs. In a disclosed embodiment, the present invention comprises the synthesis of ligands and aptamers that selectively target the stem loop (SL1) kissing-loop complex or the extended duplex formed by the dimerization initiation site (DIS) of HIV-1 ribonucleic acid (RNA). More specifically, the present invention comprises the synthesis and use of aminoglycosides that selectively target the kissing-loop complex by facilitating or preventing either the dissociation of the kissing-loop complex, or its formation thereby inhibiting replication of the HIV-1 RNA. Another disclosed embodiment is a composition comprising such aminoglycosides and to a method of using these compositions in medical therapeutic and diagnostic procedures.

The present invention is a compound with the following generic formula:

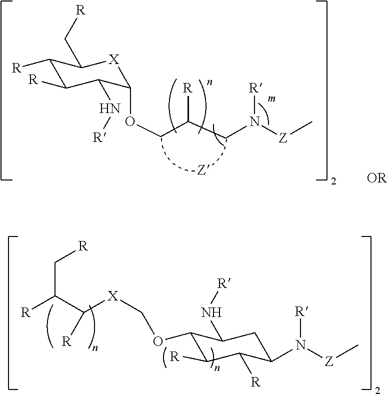

wherein X is a carbon, an oxygen, nitrogen or sulfur atom; wherein Z and Z′ are selected from the group consisting of an alkyl chain optionally containing amine or hydroxyl groups; and wherein R and R′ can be the same or different and are independently selected from the group consisting of amine or hydroxyl groups. “n, m” is a number between 1 and 5.

The kissing-loop complex that initiates dimerization of genomic RNA is crucial for HIV-1 replication. Targeting this dimerization step will lead to a new way to fight HIV and thus lead to new therapy for AIDS. The present application describes the synthesis and activities of molecules interacting with the dimerization initiation site (DIS).

High resolution structures of DIS/aminoglycoside complexes provide the necessary basis to rationally design aminoglycoside derivatives or mimics that would prevent replication by cleaving, cross-linking, or covalently modifying the viral RNA. Based on the structural data, various aminoglycosides derivatives, dimeric or not, have been designed and their synthesis achieved. New compounds have been produced and their binding to DIS confirmed.

Aminoglycoside bindings were determined by non-denaturizing gel migration, by protection toward dimethylsulfate (DMS) alkylation of RNA and toward specific cleavage induced by lead. The ability of aminoglycosides to stabilize the loop-loop interaction of DIS was investigated through competition experiments monitoring the exchange of preformed RNA homodimers (1-615/1-615) with the same but shorter RNA (1-311), in the presence, or not, of aminoglycosides.

After determination of the structure of the DIS motif, the similarities to the structure of bacterial ribosomal A site suggest that the kissing-loop complex and the extended duplex can be targeted by aminoglycoside derivatives. The binding of natural aminoglycosides to the DIS was confirmed and proved specific regarding both the aminoglycoside family and the RNA subtype. This feature led to the rationale for blocking the DIS dimer and suggests that dimeric molecules are able to selectively bind into cavities present in the DIS kissing-loop complex. The structure explains the specificity for 4,5-disubstituted 2-deoxystreptamine (DOS) derivatives and for subtype A and subtype F kissing-loop complexes and extended duplex, and provides a strong basis for rational drug design.

As a consequence of the different topologies of the kissing-loop complex and the bacterial ribosomal A site, aminoglycosides establish more contacts with HIV-1 RNA than with 16S RNA. Binding of neomycin, paromomycin and lividomycin strongly stabilized the kissing-loop complex by bridging the two HIV-1 RNA molecules.

Accordingly, it is an object of this invention to provide molecules that target the kissing-loop complex, which initiates dimerization of genomic HIV-1 RNA, and/or the corresponding extended duplex, thereby preventing replication of HIV-1 RNA.

Another object of the present invention to synthesize and use aminoglycosides that selectively target the kissing-loop complex by preventing either the dissociation of the kissing-loop complex, or its reformation, as well as the corresponding extended duplex, thereby inhibiting replication of the HIV-1 RNA.

Another object of the present invention is to design aminoglycoside derivatives or mimics that would prevent replication of HIV-1 RNA by cleaving, cross-linking, or covalently modifying the viral RNA.

A further object of the present invention is to provide an anti-HIV-1 drug for inhibiting the replication of HIV-1 RNA.

A further object of the present invention is to provide an anti-HIV-1 drug which has few or no unwanted or undesirable side effects.

These and other objects, features and advantages of the present invention will become apparent after a review of the following detailed description of disclosed embodiments and the appended drawing and claims.

### BRIEF DESCRIPTION OF THE DRAWINGS

FIG. 1A depicts the DIS in the genomic HIV-1 RNA; the sequence corresponds to sub-type A (black: modifications for sub-type B).

FIG. 1B depicts the secondary structure of the “Kissing Complex”.

FIG. 1C depicts the extended duplex secondary structure.

FIG. 2 shows X-ray structure of subtype-F DIS kissing-loop (PDB ID 1M28) showing A280 from each strand stacked inside the helix and the four bulged-out adenines forming a A272/A273/A273*/A272* stacking (left); X-ray structure of subtype-B DIS extended duplex (PDB ID 1ZCI) with a four bulged-out adenine stacking A273/A272/A272*/A273* while A280 from each strand is stacked inside the helix (right).

FIG. 3 shows a stereoscopic view of neomycin docked into the DIS ‘kissing complex’.

FIG. 4 is a model view of neomycin within the DIS kissing complex, after suppression of the front part of the RNA.

FIG. 5 depicts the structures of neomycin, paromomycin, ribostamycin, and neamine.

FIG. 6 shows the synthesis of fully protected, but differentiated, neamine derivatives.

FIG. 7 shows the synthesis of neamine derivatives carrying a chain at position 1.

FIG. 8 depicts some of the compounds derived from D-glucosamine (D-GlcN) or from 2-deoxystreptamine (2-DOS).

FIG. 9 depicts the synthesis of compounds derived from D-glucosamine.

FIG. 10 depicts protection by various aminoglycosides, including a neamine dimer toward lead induced DIS cleavage.

FIG. 11A depicts DIS-neamine and FIG. 11B depicts DIS-neamine dimer crystals.

FIGS. 12A, 12B and 12C depict stereoviews of X ray DIS structures. FIG. 12A shows the superposition of free DIS structure with the ribostamycine complex; magnesium (central sphere) is only in the free form. FIG. 12B shows part of the electronic density map for the neamine-DIS complex. FIG. 12C shows part of the electronic density map for the neomycin-DIS complex.

FIG. 13 shows the stabilization of the loop-loop interaction competition between RNA of different sizes followed by gel migration. Curves showed the percent of RNA heterodimer (1-615/1-311) formation starting from homodimer (1-615/1-615) function of aminoglycoside concentration.

FIG. 14 shows stabilization of the kissing complex by dimer and aminoglycoside antibiotics. Radiolabeled RNA 1-615 was incubated in dimerization buffer, and a 5-fold excess of unlabeled RNA1-311 was added to the solution in absence or in presence of dimer or aminoglycosides (see examples of gels in the lower insert: lane C, control dimer without aminoglycoside; d, m, and h stand for dimer, monomer and heterodimer, respectively). Each band on the gel was quantified using the MacBAS software (Fuji) and percentage of heterodimer was plotted as a function of incubation time.

FIG. 15 depicts 4,5-disubstituted DOS derivatives.

FIG. 16 depicts 4,6-disubstituted DOS derivatives.

FIGS. 17a and 17b depict the structure of the ribostamycin/DIS complex.

FIGS. 17c and 17d provide details of the DIS-ribostamycin complex.

FIG. 18 depicts a schematic drawing summarizing all neomycin-RNA contacts. (w) and (K) spheres represent water molecules and a potassium cation, respectively. The two RNA strands of the kissing-loop complex are represented.

FIG. 19A superimposes the ribosomal A site-paromomycin complex with the

DIS-neomycin complex. FIG. 19B superimposes the paromomycin in the A site with neamine bound to the DIS loop-loop complex. Rings Ito IV are labeled, whereas Ring V of lividomycin is not. The r.m.s.d. for rings I and H ranges from 0.08 to 0.25 Å.

FIG. 20 depicts stereo views showing a superpostion of the DIS/neomycin complex with the ribosomal A site/tobramycin complex. The steric hinderance between the DIS RNA and the tobramycin depicted in the close-up view below the structure.

FIG. 21 shows in vitro binding of aminoglycosides to subtype F and subtype A DIS and the inhibition of lead (II)-induced cleavage of a 23 mer subtype F DIS RNA by aminoglycosides. Nea, Ribo, Paro, Livi, Tobra, Kana, Genta represent neamine, ribostamycin, paromomycin, lividomycin, tobramycin, kanamycin and gentamycin, respectively. Numbers correspond to μM concentration of the aminoglycoside. −Pb and +Pb correspond to control lanes without aminoglycoside, without and with lead acetate, respectively.

FIG. 22A shows in vitro binding of aminoglycosides to subtype F and subtype A DIS and the inhibition of lead-induced cleavage of MAL RNA1-615. Lanes 1-4 correspond to 0, 10, 50 and 100 μM aminoglycoside. (−) corresponds to an extension control performed in the absence of lead acetate. FIG. 22B represents DMS footprinting of aminoglycosides on MAL RNAI-615. Aminoglycoside concentrations and control lanes are as in FIG. 22A.

FIG. 23 depicts the ‘kissing loop’ complex and its mechanism of dimerization.

FIG. 24 shows stabilization of the kissing-loop complex by 4,5-disubstituted DOS. Radiolabeled RNA 1-615 was incubated in dimerization buffer, and unlabeled RNA1-311 was added to the solution in absence or in presence of aminoglycoside, as indicated above the gel. Lane C is a control dimer without aminoglycoside.

FIG. 25 shows viral replication of wild-type and mutant molecular clones. H9 cells were infected with the parental NL4.3 virus or with the indicated mutants, and viral replication was monitored by measuring RT activity in the culture supernatants every 2-3 days.

FIGS. 26A and 26B show aminoglycoside-induced DIS protection in CEM×174 cells and in virions. FIG. 26A depicts methylation of the genomic RNA in infected cells and in virions. Lane (−), extension control without DMS; lane C, DMS treatment without aminoglycoside; lanes L, N, and P: DMS treatment in the presence of lividomycin, neomycin and paromomycin, respectively. Sequence lanes (U, A, C, and G) were run in parallel to identify the modified nucleotides.

FIG. 26B show the quantification of the gels of FIG. 26A, and modification of A280, normalized to that of A₂₇₂, and compared to that obtained in the absence of aminoglycoside (which was arbitrarily set to 1 for each HIV-1 subtype).

### DETAILED DESCRIPTION OF THE INVENTION

The present invention may be understood more readily by reference to the following detailed description of specific embodiments included herein. Although the present invention has been described with reference to specific details of certain embodiments thereof, it is not intended that such details should be regarded as limitations upon the scope of the invention. The entire text of the references mentioned herein are hereby incorporated in their entireties by reference.

To provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value.

The present description refers to a number of chemical terms and abbreviations used by those skilled in the art. Nevertheless, definitions of selected terms are provided for clarity and consistency.

As used herein, the term “anti-HIV-1 drug” refers generally to the embodiments of the disclosed invention.

As used herein, the term “aptamer” refers to reagents generated in a selection from a combinatorial library (typically in vitro) wherein a target molecule, generally although not exclusively a small molecule such as a metabolite or a drug, or a protein or nucleic acid, is used to select from a combinatorial pool of molecules, generally although not exclusively oligonucleotides, those that are capable of binding to the target molecule. The selected reagents can be identified as primary aptamers. The term “aptamer” includes not only the primary aptamer in its original form, but also secondary aptamers derived from (i.e., created by minimizing and/or modifying) the primary aptamer. Aptamers, therefore, must behave as ligands, binding to their target molecule. Aptamers that bind small molecules have been shown to undergo conformational changes upon interactions with their cognate ligands. A reporter fluorophore introduced into an aptamer in a region known to undergo conformational change can lead to a change in fluorescence intensity or polarization after the binding event. The use of aptamers in RNA constructs is described for instance in U.S. Pat. Nos. 6,458,559 and 6,706,481 which are hereby incorporated herein in their entirety by reference. The aptamer may also select for other types of target molecules, such as organic molecules such as pharmaceuticals, carbohydrate or other biomolecules. The role of the aptamer generally is to allow for a change in the conformation of the aptamer containing RNA of a helical stacking domain such that its binding sites become stereochemically better disposed to binding interaction.

Despite the efficiency of highly active antiretroviral therapy, the emergence of resistant strains and the side effects of the current treatments highlight the need for new inhibitors of HIV-1 replication. In addition to viral and cellular proteins, the genomic RNA itself has been proposed as a drug target (Wilson et al., Curr. Med. Chem. 2000, 7:73-98). For instance, molecules targeting the trans activating region (TAR) (Hamy et al., Proc. Natl. Acad. Sci. USA 1997, 94:3548-3553; Hwang et al., 2003; Wang et al., J. Biol. Chem. 1998, 278:39092-39103), the packaging signal (McPike et al., Bioorg. Med. Chem. 2002, 10: 3663-3672; McPike et al., Nucleic Acids Res. 2002, 30:2825-2831), the frameshifting signal (Hung et al., J. Virol. 1998, 72:4819-4824), and the rev responsive element (RRE) (Wang et al., Biochemistry 1997, 36:768-779; Zapp et al., Cell 1993, 74:969-978) have been selected in vitro. Aminoglycosides have been shown to bind to several of these sites (McPike et al., Bioorg. Med. Chem. 2002, 10: 3663-3672; McPike et al., Nucleic Acids Res. 2002, 30:2825-2831); Wang et al., J. Biol. Chem. 1998, 278:39092-39103; Wang et al., Biochemistry 1997, 36:768-779; Zapp et al., Cell 1993, 74:969-978), but in most cases, the selectivity and specificity of binding were not addressed or poorly understood.

We took advantage of the structural similitude we identified between the crystal structures of the bacterial ribosomal A site and the HIV-1 kissing-loop complex and the extended duplex (Ennifar et al., J. Biol. Chem. 2003, 278:2723-2730) to target the DIS with aminoglycosides that are known to specifically interact with the bacterial A site. Indeed, binding of aminoglycosides to the kissing-loop complex could be predicted from its crystal structure, characterized by the first two purines of the DIS loop bulging out (Ennifar et al., Nat. Struct. Biol. 2001 8:1064-1068), but not from its NMR structures (Baba et al., J. Biochem. (Tokyo) 2005, 138:583-592; Mujeeb et al., Nat. Struct. Biol. 1998, 5:432-436), in which these purines are buried in the structure. The crystal structures of the DIS/aminoglycoside complexes we report here confirm the strong similitude of the kissing-loop complex with the ribosomal A site, and support the biological relevance of the flipped out conformation of the first two purines of the DIS loop. Strangely enough, several contacts are specific for the kissing-loop complex, and 4,5-disubstituted DOS build more direct interactions with the kissing-loop complex than with the A site.

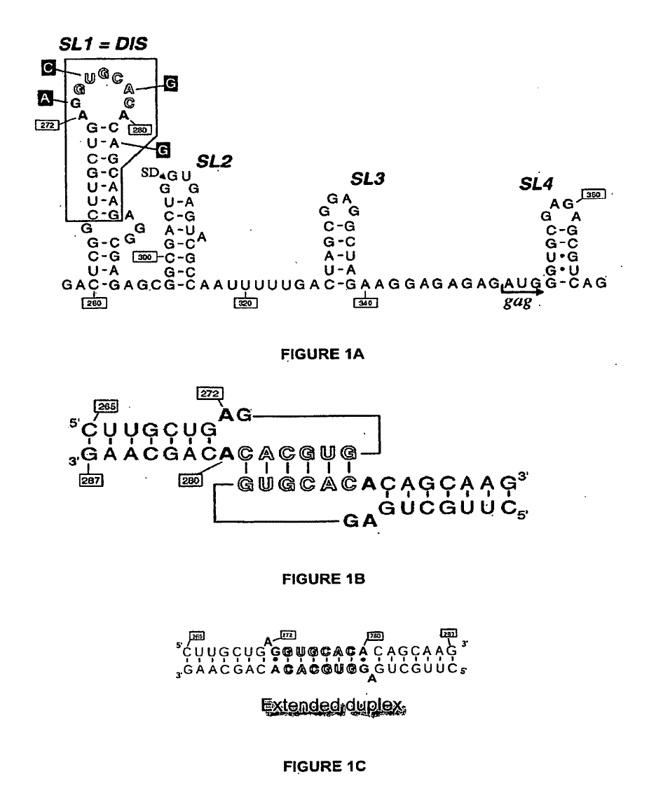

Interestingly, our crystal structures and biochemical experiments revealed that binding of aminoglycosides to the DIS is specific regarding both the aminoglycoside family and the RNA subtype. In contrast with 4,5-disubstituted DOS, 4,6-disubstituted DOS, which also bind to the bacterial ribosomal A site, do not specifically bind to the HIV-1 kissing-loop complex. Our crystal structures combined with modeling strongly suggest that residue A₂₇₈ prevents binding of 4,6-disubstituted DOS to the kissing-loop complex. Indeed, the structurally equivalent position in the ribosomal A site is U₁₄₀₆ (FIG. 1d), and it has been shown that substituting an A for U₁₄₀₆ in the 16S ribosomal RNA confers resistance to 4,6-disubstituted DOS, but not to 4,5-disubstituted DOS (Pfister et al., Chembiochem. 2003, 4:1078-1088; Recht et al., Chemother. 2001, 45:2414-2419).

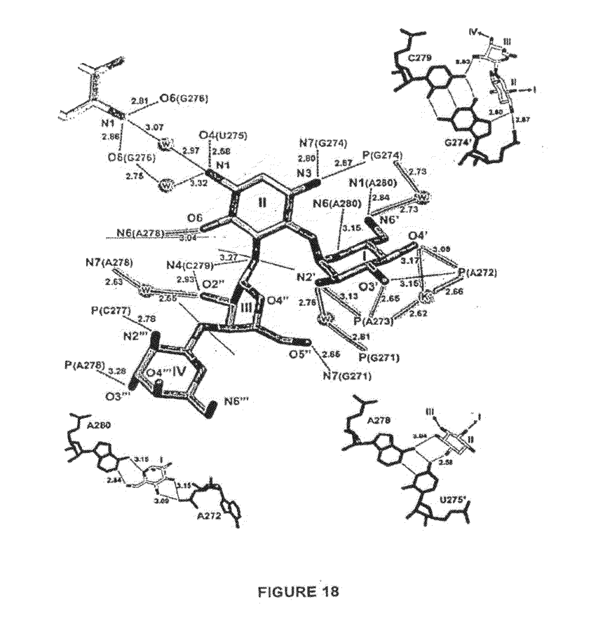

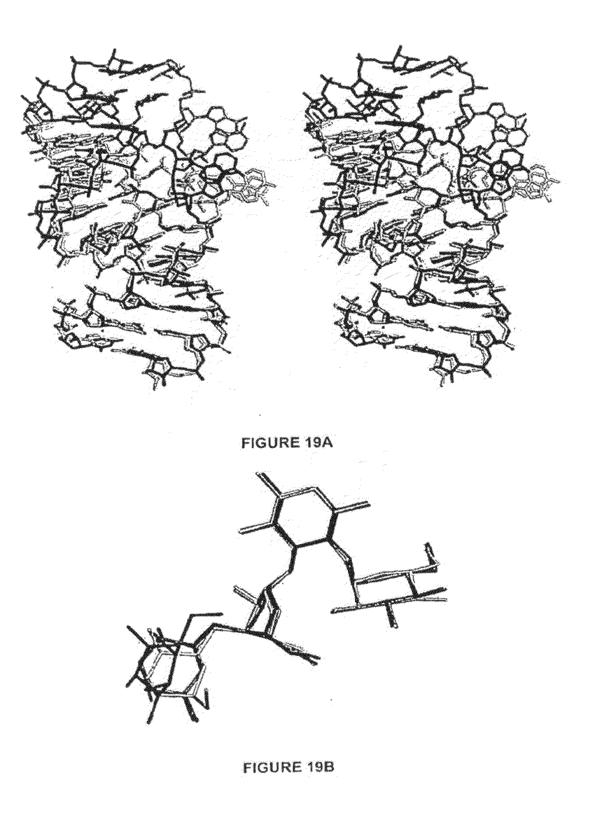

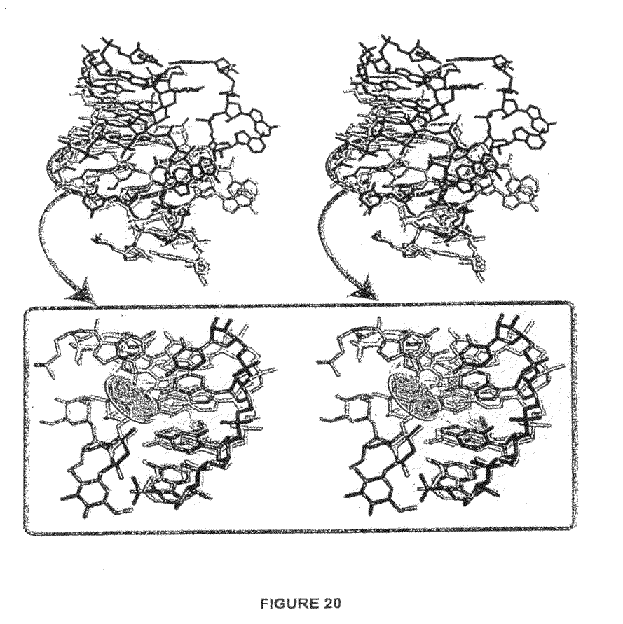

4,5-disubstituted DOS bind to subtype A and subtype F, but not to subtype B DIS. Our X-ray structures of the subtype F DIS complexed with 4,5-disubstituted DOS demonstrate that this subtype specificity is linked to the identity of the second nucleotide in the DIS self-complementary sequence. This nucleotide is a uridine (U₂₇₅) in all HIV-1 subtypes, except in subtypes B and D. The corresponding nucleotide in the bacterial ribosomal A site is also a uridine (U₁₄₉₅) (FIG. 19-20). In the complexes of the subtype F kissing-loop complex with 4,5-disubstituted DOS, atom O4 of U₂₇₅ makes a hydrogen bond with the amino group at position 1 of ring II of the aminoglycoside (FIG. 18). In the subtype B kissing-loop complex, the amino group at position 4 of C₂₇₅ would clash with the aminoglycoside amino group, thus preventing binding. In line with this interpretation, substituting a C or an A, but not a G, for U₁₄₉₅ in the E. coli A site RNA prevents paromomycin binding (Recht et al., J. Mol. Biol. 1996, 262:421-436), whereas substituting a C residue for U₁₄₉₅ in Mycobacterium smegmatis 16S RNA confers resistance to both 4,5-disubstituted and 4,6-disubstituted aminoglycosides (Pfister et al., Chembiochem. 2003, 4:1078-1088). Interestingly, our crystal structures indicate that substituting a hydroxyl for the amino group at position 1 of ring II of 4,5-disubstituted DOS should allow to target subtype B HIV-1 strains.

The structures predict that 4,5-disubstituted DOS should not bind to the kissing-loop complex of HIV-1 strains belonging to subtype C of group M (main) and to group O (outlier). In these strains, A₂₈₀, which form a pseudo Watson-Crick base-pair with the aminoglycoside ring I (FIG. 18), is replaced by a uridine or a cytosine, respectively. If these residues are unpaired and stacked inside the structure, as A₂₈₀ in the kissing-loop structure of subtypes A&G, B&D, and F&H, it should be possible to modify ring I in order to specifically target these strains. However, in these strains, nucleotide 280 can potentially form a Watson-Crick base-pair with residue 272, reducing the DIS loop to seven nucleotides. If this base-pair does form, it would be impossible to target the corresponding kissing-loop complexes with 4,5-disubstituted aminoglycosides. Thus, it would be interesting to solve the structure of these kissing-loop complexes.

The selectivity of binding with respect to the aminoglycoside family and to the HIV-1 subtype clearly show that binding of 4,5-disubstituted DOS to the HIV-1 kissing-loop complex does not only rely on non specific electrostatic interactions, but also on specific contacts. Our crystal structures clearly indicate that the binding specificity is mainly driven by rings I and II, and to a lesser extend by ring III of 4,5-disubstituted DOS, which interact with the RNA bases and the particular sugar-phosphate backbone motif of the kissing-loop complex. At the opposite, rings IV and V mainly contribute to binding by interacting with phosphate groups in a regular A type conformation. However, our biochemical experiments demonstrate that these interactions are essential to target the DIS when multiple non-specific targets are also present.

Indeed, the in situ footprinting experiments demonstrated that 4,5-disubstituted DOS with four or five rings are able to target the DIS in infected cells and in virions. These experiments demonstrate that the DIS is accessible in vivo, a prerequisite for a drug target. This is the first demonstration that aminoglycosides specifically bind to HIV-1 genomic RNA in cell culture, and to the best of our knowledge, the only direct demonstration that a small ligand directed against HIV-1 RNA does bind to its predicted target in infected cells and/or in viral particles.

Binding of 4,5-disubstituted DOS to the DIS appears to stabilize the kissing-loop complex. The compounds were predicted to bind to the kissing-loop complex rather than to the monomeric form of the DIS. At odds with our results, neomycin and paromomycin were also reported to bind to the subtype B DIS loop under ionic conditions in which the monomeric form of HIV-1 RNA prevails (McPike et al., Bioorg. Med. Chem. 2002, 10: 3663-3672; McPike et al., Nucleic Acids Res. 2002, 30:2825-2831). However, as these experiments were conducted at very low ionic strength and in the absence of multivalent cations, this binding was most likely driven by unspecific electrostatic interactions, in keeping with the numerous binding sites observed in the HIV-1 packaging signal under these conditions (McPike et al., Bioorg. Med. Chem. 2002, 10: 3663-3672; McPike et al., Nucleic Acids Res. 2002, 30:2825-2831).

These studies demonstrate that aminoglycosides can specifically target the DIS of the HIV-1 genomic RNA in cells and in virions. High resolution structures of DIS/aminoglycoside complexes provide the necessary basis to rationally design aminoglycoside derivatives that would prevent replication by cleaving, cross-linking, or covalently modifying the viral RNA. They also allow identification of aminoglycoside mimics that would make similar interactions with the kissing-loop complex but would be easier to modify.

### Structural Model

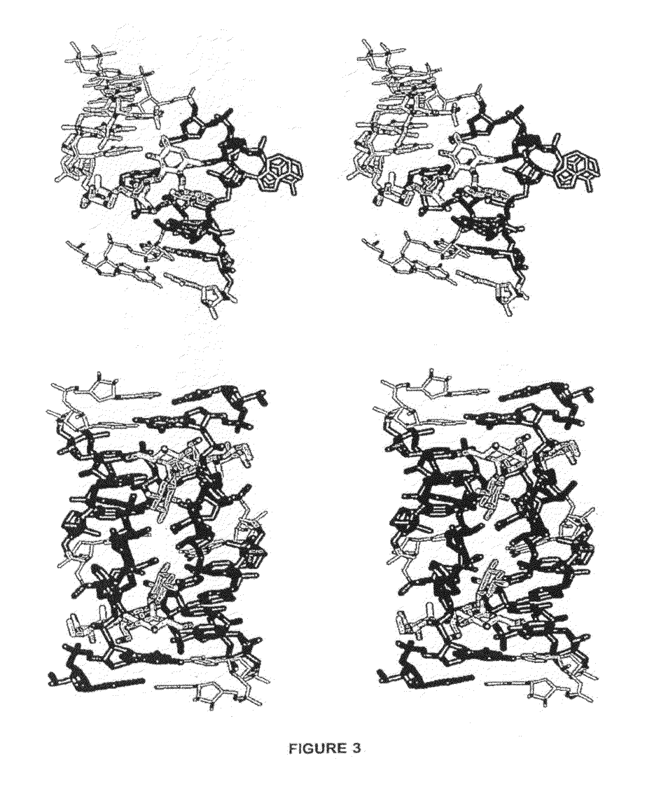

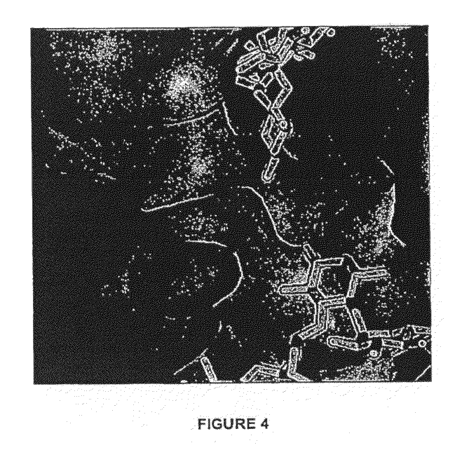

Based on the known structure of ribosomal A-site bonded to paromomycin (Vicens et al., Structure 2001, 9:647-658), a model of aminoglycoside bonded to the DIS kissing-complex is shown. This model showed that 2 neomycin moieties are facing each other in each DIS cavity at around 4-6 A (FIGS. 3 and 4).

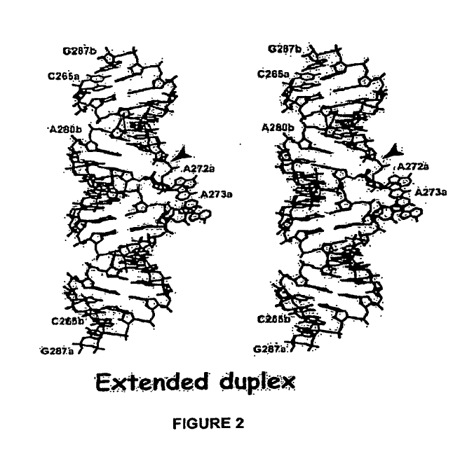

After determination of the structure of the DIS motif, the similarities to the structure of bacterial ribosomal A site suggest that the kissing-loop complex can be targeted by aminoglycoside derivatives. The binding of natural aminoglycosides to the DIS was confirmed and proved specific regarding both the aminoglycoside family and the RNA subtype. This feature led to the rationale for blocking the DIS dimer and thus HIV replication with dimeric molecules being able to selectively bind into both cavities (FIG. 2).

Further, crystallographic results revealed an unexpected structural and sequence similarity with the A-site of bacterial ribosomes, known to be the target of aminoglycoside antibiotics, interfering with translation accuracy.

Owing to its strong similarity with the bacterial ribosomal A site (Ennifar et al., J. Biol. Chem. 2003, 278:2723-2730), the DIS will bind aminoglycosides. This hypothesis was further supported by molecular modelling and solution studies (Ennifar et al., J. Biol. Chem. 2003, 278:2723-2730) and recently proved by biological and co-crystallization studies (Ennifar et al., Nucleic Acids Res., 2006, 34:2328-2339). The latter results showed that most of the specific interactions are localized on the neamine part of these antibiotics. On this basis, the design, synthesis and study of dimeric neamine derivatives as new potent anti-HIV agents was made, resulting in the first synthesis of such a dimer and its binding to DIS.

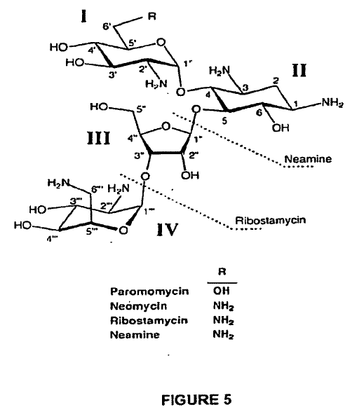

Synthesis Overview Based on this model (FIG. 4), several series of molecules, dimeric or not, analogs to neomycin were synthesized (FIG. 5) and designed to bind to the DIS kissing-complex.

In one series (A), the model led to the design of neamine derivatives where the amino group at the position 1 in cycle II (FIG. 5) are linked together with a chain of appropriate length and functionality.

In another series (B), this amino group was used to connect a neamine derivative with chains of various length and functionality.

In a third series (C), simpler molecules mimicking neamine were designed with a chain of appropriate length and functionality. Choosing the right motif allows the molecule to keep an appropriate conformation and some rigidity while giving some flexibility compared to the precedent series. Functional groups were selected to keep the interactions essential for binding to DIS.

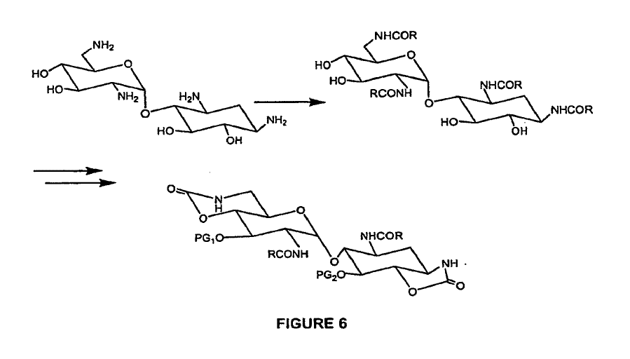

The two A and B series were prepared from the same building block obtained from neamine. The latter was obtained by partial hydrolysis of commercially available neomycin through a protocol adapted from the known procedure (Dutcher et al., J. Am. Chem. Soc. 1952, 74, 3420; Byron et al. J. Am. Chem. Soc. 1951, 73, 2794). The four amino groups in neamine were then protected through known procedures (Kociensky, Protecting Groups, 3^{rd} Ed. Thieme 2004). From these tetra N-protected derivatives, we were able to differentiate two amino groups by intramolecular selective reactions with adjacent hydroxyl groups. Protection of the remaining hydroxyl groups led to fully protected neamine derivatives (FIG. 6).

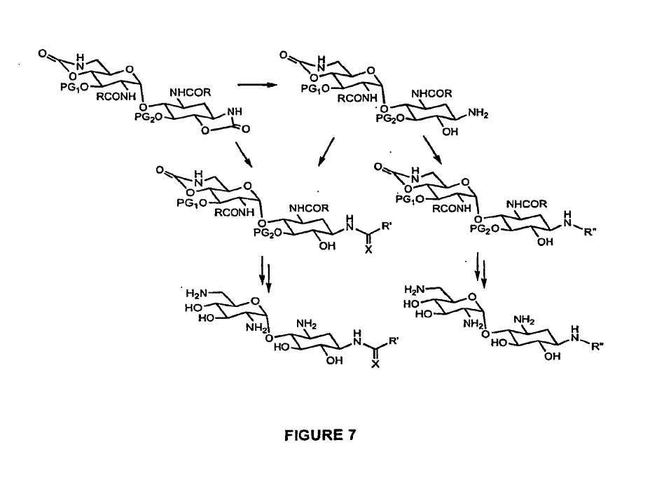

Controlled conditions allowed for either selective opening and introduction of the chain at position 1 or selective liberation of the required amino group at position 1 (FIG. 7—top). In the latter case, various protecting groups were then removed with the appropriate conditions, leading to the above-mentioned series of neamine derivatives, dimeric or not (FIG. 7 - bottom).

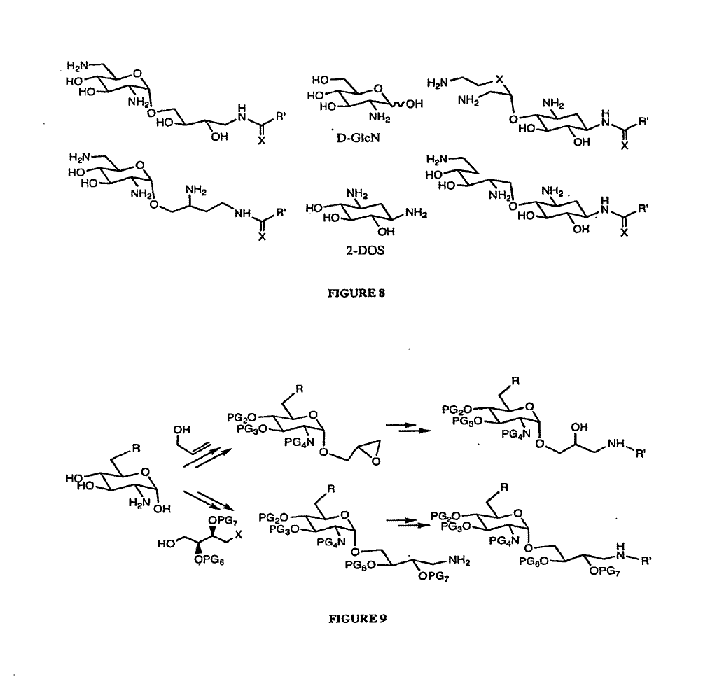

In the C series, a huge number of compounds can be designed. They could be synthesized either from D-glucosamine (D-GIcN) or from 2-deoxystreptamine (2-DOS) (FIG. 8).

A series derived from D-glucosamine were obtained (FIG. 9). Glycosylation with allyl alcohol or tartaric derivatives yielded the corresponding glycosides, which upon functional group manipulations gave glucosamine derivatives carrying at their anomeric position a hydroxyamino or a dihydroxyamino chain. The amino group in the latter can then be used to anchor another chain or to link together 2 units.

After deprotection, various glucosamine derivatives, dimeric or not, were obtained.

### Detailed Synthesis

One strategy to obtain such dimers is to start from neamine, easily produced from the commercially available neomycin sulfate (Dutcher et al., J. Am. Chem. Soc., 1952, 74:3420; Byron et al., J. Am. Chem. Soc., 1951, 73:2794) (Scheme 1).

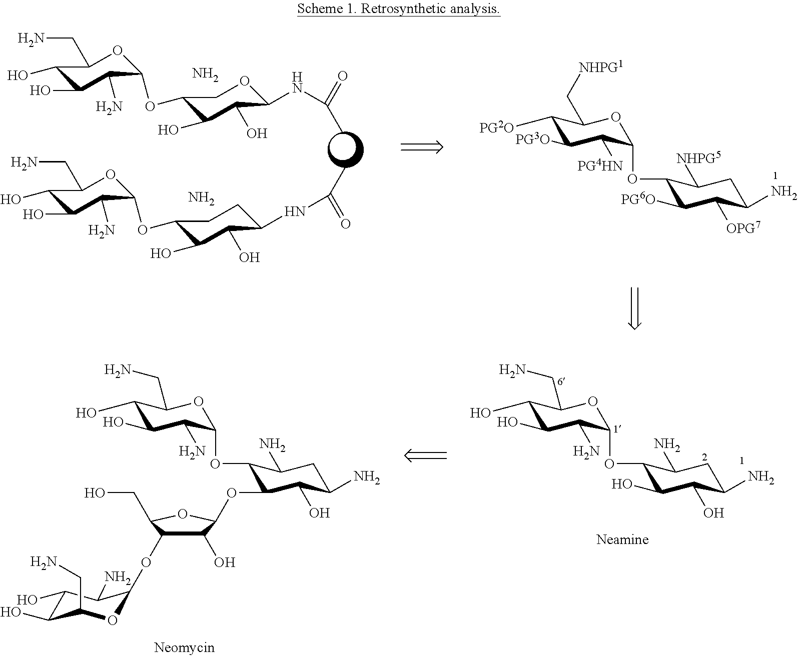

This requires selectively protecting all the neamine functions except amine N1 in order to graft a spacer on it. The optimal spacer length was evaluated from the crystallographic structures to be a four carbon atom (five bonds) chain. To maintain and even extend the interactions due to the N1 amino group, amide linkages were preferentially chosen; however, other linkages can be used. Although there is renewal in aminoglycoside chemistry mainly driven by the fight against antibiotic resistance (Li et al., Anti-Infective Agents in Medicinal Chemistry 2006, 5:255-271; Yajima et al., Bioorg. Med. Chem. 2006, 14:2799-2809; Gao et al., Angew. Chem. Int. Ed. 2005, 44:6859-6862; Agnelli et al., Angew. Chem. Int. Ed. 2004, 43:1562-1566; Rege et al., J. Am. Chem. Soc. 2004, 126:12306-12315; Venot et al., ChemBioChem 2004, 5:1228-1236), only a few approaches aiming at distinguishing the N1 amine from the other amine and alcohol functions were described. Mobashery et al. prepared a protected neamine derivative having only amine N1 and alcohol 06 free in 11 steps with an overall yield of 29% (Haddad et al., J. Am. Chem. Soc. 2002, 124:3229-3237). Chang et al. synthesized a neamine derivative totally protected, but having amine N1 protected by a BOC group instead of the azido group carried by the other three amines. This approach required 8 steps with an overall yield of 12% and is based on a selective Staudinger reaction, but the selectivity remained poor and a side product was not separable from the expected product (Li et al., Org. Lett. 2005, 7:905-910).

To have a flexible, but still short sequence, selective formation of cyclic carbamates owing to the vicinity of hydroxyl and amino groups was obtained (Agami et al., Tetrahedron 2002, 58, 2701-2724; Dyen et al, Chem. Rev. 1967, 67:107-246; Ohno et al., Org. Lett. 2000, 2 :2161-2163; Junquera et al., Tetrahedron 1996, 52:5545-5548) and later, on selective opening of these cyclic carbamates (Agami et al., Tetrahedron 2002, 58 :2701-2724; Dyen et al., Chem. Rev. 1967, 67:107-246), expecting that a strained carbamate opens up more readily than an unstrained one (Scheme 2).

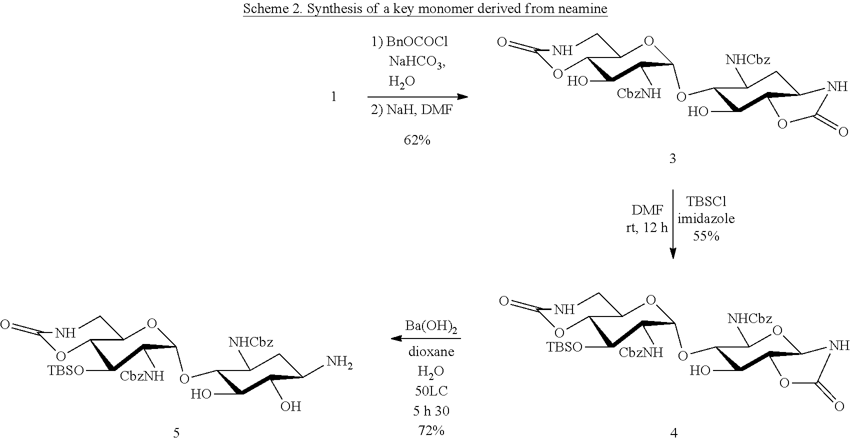

Neamine 1 was converted to its known N-tetra(carbobenzyloxy) derivative 2 (Park et al., J. Am. Chem. Soc. 1996, 118:10150-10155; Canas-Rodriguez et al., Carbohydr. Res. 1977, 58:379-385; Kumar et al., J. Org. Chem. 1978, 43:3327; Umezawa et al., Bull. Chem. Soc. Jpn. 1969, 42:537-541), other protecting groups being less effective in the following sequence. Compound 3, having two amines (N1, N6′) distinguished from the two others (N3, N2′), was then prepared from 2 by selective deprotonation under carefully control conditions. The formation of a third cyclic carbamate between alcohol 3′ and amine 2′ had to be avoided. The more reactive 3′ alcohol was then protected in order to avoid formation of the third possible carbamate in the next steps.

Various silyl groups can be introduced but the TBS group proved to be the preferred compromise between convenience and yield, but an excess of TBSCI and imidazole were necessary to obtain a satisfactory yield. An important step was the selective opening of the trans-fused five membered carbamate. As cyclic carbamates are known to decarboxylate under basic conditions [11, 16], careful conditions were therefore required. Treatment of compound 4 by barium hydroxide (Horton et al., Carbohydr. Res. 1975, 44: 227-240; Crewe et al., Chem. Ber. 1959, 92:1195-1205) in a mixture of dioxane and water allowed for the expected selective opening with good yield. This 4-step sequence provided compound 5, protected except at amine N1 and alcohols O5 O6, in a very good overall yield (25%). The two alcohol functions could also be protected (Haddad et al., J. Am. Chem. Soc. 2002, 124:3229-3237; silyl groups could also be introduced using classical methods), but the higher nucleophilicity of the amino group led to introducing the required linker through amide bond formation.

Numerous methods have been developed for efficient amide formation, but only a few reagents are known to be selective for amine in the presence of alcohols. N-hydroxysuccinimide (NHS) esters (Kawaguchi et al., J. Antibiotics 1972, 25:695-708; Haddad et al., J. Am. Chem. Soc. 2002, 124:3229-3237; Katsarava et al., Makromol. Chem. 1986, 187:2053-2062; Tanaka et al., Bioorg. Med. Chem. Lett. 2002, 12:1723-1726) or pivaloyl anhydrides (Kocian et al., Coll. Czech. Chem. Comm. 1982, 47:1356-1366; Myers et al., J. Am. Chem. Soc. 1997, 119:656-673) are among them. Nevertheless, the dimerization reaction could be far from trivial due to competitive entropically favored intramolecular reactions between the linker and the alcohol groups adjacent to the amine, or between both ends of the linker and the amine. To avoid such problems, a rigid linker derived from fumaric acid was selected. Due to its E double bond, fumaric acid could not allow such side reactions, neither during the preparation of activated diesters, nor during the dimerization step.

The corresponding activated NHS diester 6 was prepared from fumaric acid with the trifluoroacetic anhydride method (Katsarava et al., Vysokomol. Soed. Ser. A. 1984, 26:1489-1497), which proved better than any others (Katsarava et al., Macromol. Chem. 1985, 186:939-954). The corresponding bis(pivaloyl) anhydride 7 was also easily obtained using conventional conditions (Kocian et al., Coll. Czech. Chem. Comm. 1982, 47:1356-1366; Myers et al., J. Am. Chem. Soc. 1997, 119:656-673). However, the later route required to directly react the bis(pivaloyl) anhydride 7 with neamine derivatives, this reagent being too unstable to be stored.

Compound 6: To an ice cold suspension of N-hydroxysuccinimide (1 g, 8.69 mmol) and fumaric acid (504 mg, 4.34 mmol) in freshly distilled pyridine (1.43 mL, 17.38 mmol) and chlorobenzene (5.4 mL) under an atmosphere of argon, was added dropwise with stirring trifluoroacetic anhydride (1.23 mL, 8.69 mmol). After 10 min at 0° C., the mixture was stirred 2 h at room temperature and was kept overnight at 4° C. The resulting solid was filtered and washed thoroughly with ethanol. Recrystallisation from acetonitrile furnished a brown product (889 mg, 66%). ¹H NMR (300 MHz, DMSO) □=2.87 (s; 4H ; CH₂), 7.48 (s; 1H ; CH); ¹³C NMR (75 MHz, DMSO) □=26.0 (CH₂), 132.3 (CH═CH), 160.4 (O—CO—CH═), 170.3 (CH₂—CO—N); HRMS ES+ Calcd for C₇₄H₁₀₁N₈NaO₂₄Si₂ (M+Na⁺) m/z=333.0329, found 333.0319.

With these reagents in hand, the dimerization step was performed (Scheme 3).

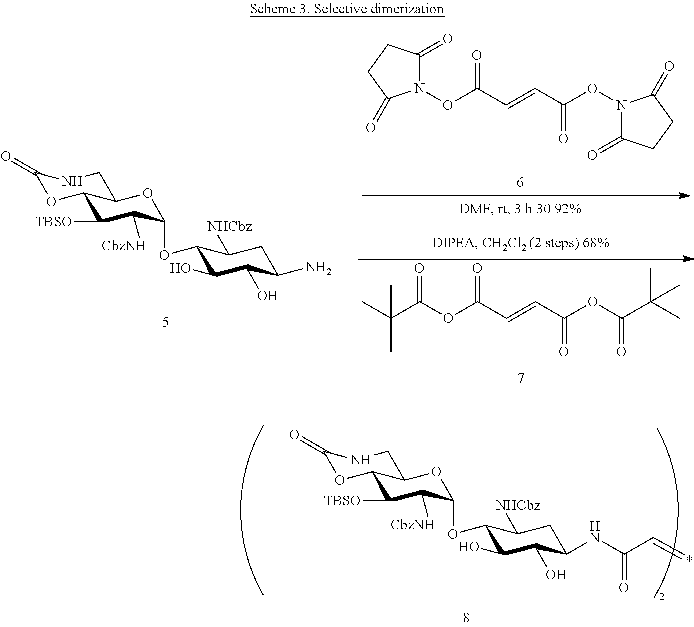

Two equivalents of compound 5 were thus reacted in DMF with one equivalent of the bis activated ester 6 or 7. As expected, the free amine function of the protected neamine 5 selectively reacted with the NHS diester 6, providing the expected dimer 8 in an excellent yield of 92%. With the bis(pivaloyl) anhydride 7, the dimer was also selectively obtained but with a lower yield.

Dimer 8: A solution of 5 (2.452 g, 3.35 mmol) and 6 (486 mg, 1.52 mmol) in anhydrous DMF (23 mL) under an atmosphere of argon was stirred for 3 h20 at room temperature. The reaction mixture was concentrated and treated with ice cold water. The resulting white solid was filtered and washed with water, then purified on a column (SiO₂, 9:1 then 6:4 CH₂Cl₂/MeOH) to furnish a white solid (2.147 g 92%). Mp: 240° C. (dec); [α]_{D}²⁰+3 (c 1.1, Py); IR(KBr): υ=3500, 2950, 1701 1650, 1522, 1457, 1405, 1259 cm⁻¹; ¹H NMR (500 MHz, pyridine-d5) δ: 0.29 (s, 3H, CH₃ TBS), 0.48 (s, 3H, CH₃ TBS), 1.05 (s, 9H, tBu TBS), 1.86 (ddd, H2ax, J_{2ax-2eq}=J₂ₐₓ₋₁=J₂ₐₓ₋₃=12.3 Hz), 2.48 (m, H2eq), 3.29 (dd, H6′ax, J_{6′ax-6′eq}=J_{6′ax-5}=10.1 Hz), 3.50 (m, H6′eq), 3.87 (m, 2H, H5 H6), 4.08 (m, H4), 4.15 (dd, H4′, J_{4′-5′}=J_{4′-3′}=8.7 Hz), 4.26 (m, H3), 4.40 (m, H1), 4.52 (m, H2′ H3′), 4.73 (m, H5′), 5.18 (d, 1H, J=12.4 Hz, CH₂—Ar Cbz2′), 5.24 (d, 1H, J=12.6 Hz, CH₂—Ar Cbz3), 5.45 (d, 1H, J=12.4 Hz, CH₂—Ar Cbz2′), 5.75 (d, 1H, J=12.6 Hz, CH₂—Ar Cbz3), 6.20 (s, H1′), 7.21 (s, OH6), 7.33 (m, 5H, Ar), 7.50 (m, 5H, Ar), 7.56 (s, 1H, CH═CH), 7.67 (s, OH5), 8.29 (d, NH6′, J_{NH-6}′=4.4 Hz), 8.52 (d, NH3, J_{NH-3}=9.1 Hz), 8.64 (d, NH2′, J_{NH-2}′=8.5 Hz), 9.38 (d, NH1, J_{NH-1}==7.8 Hz); ¹³C NMR (125 MHz, pyridine-d5) δ −4.7 (CH₃ TBS), −3.6 (CH₃ TBS), 18.6 (C tBu), 26.2 (Me tBu), 35.8 (C2), 44.0 (C6′), 51.0 (C1 C3), 57.1 (C2′), 62.5 (C5′), 66.5 (CH₂—Ar Cbz3), 66.7 (CH₂—Ar Cbz2′), 71.8 (C3′), 76.6 (C6), 78.4 (C5), 80.2 (C4′), 82.3 (C4), 100.5 (C1′), 128.1 (Ar C^{III}), 128.2 (Ar C^{III}), 128.4 (Ar C^{III}), 128.5 (Ar C^{III}), 128.8 (Ar C^{III}), 133.9 (CH═CH), 137.8 (Ar C^{IV} Cbz2′), 138.1 (Ar C^{IV} Cbz3), 153.3 (C═O six-membered carbamate), 156.9 (C═O Cbz), 157.6 (C═O Cbz), 165.0 (C═O spacer); HRMS ES+ Calcd for C₇₄H₁₀₁N₈O₂₄Si2 (M+H⁴) m/z=1541.6462, found 1541.6456.

Deprotection of carbobenzyl groups (Cbz) could not be achieved under conventional hydrogenation conditions. After extensive investigations, treatment with sodium in liquid ammonia led to both hydrogenation of the double bond and deprotection of the four Cbz groups (Scheme 4).

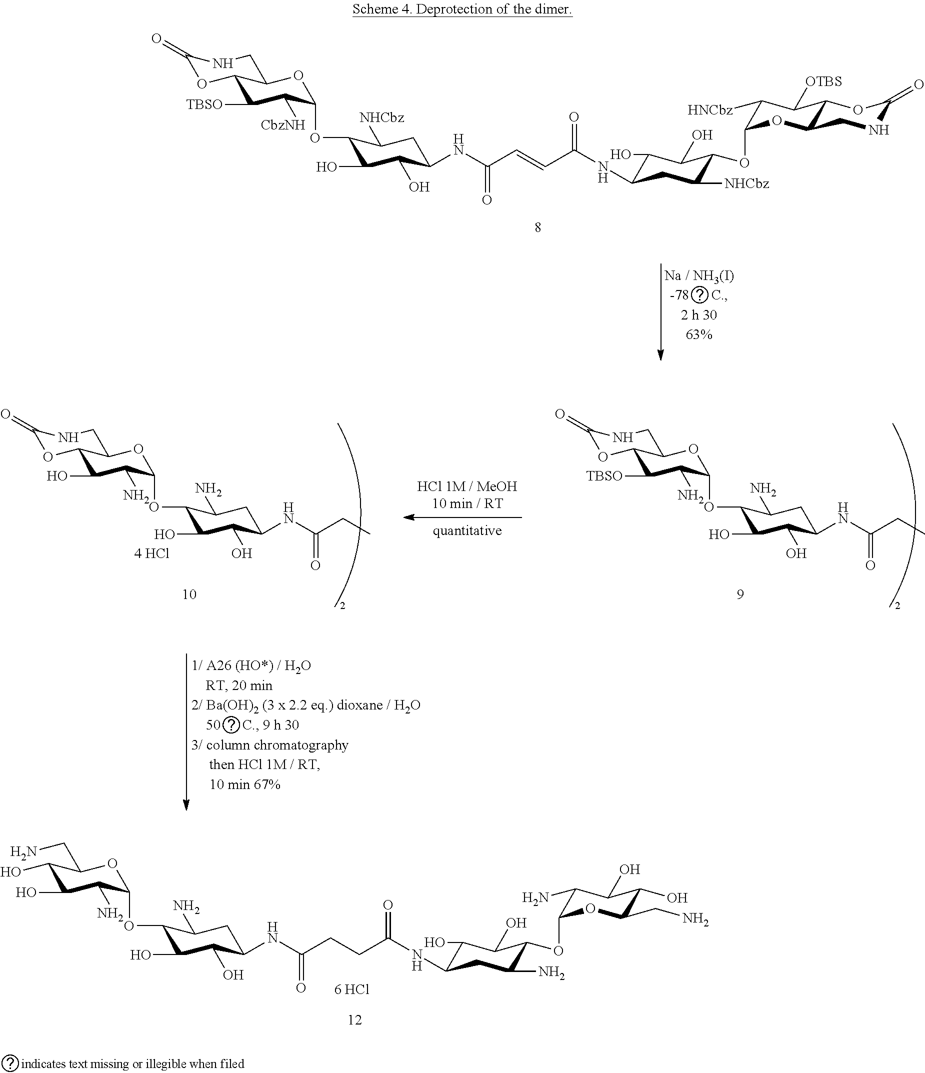

Alternative sequences can be used, and for example, diacids can be condensed in controlled conditions with compound 5 in the presence of diisopropylcarbodiimide in dichloromethane (Scheme 5). The deprotection can be achieved in carefully controlled basic conditions.

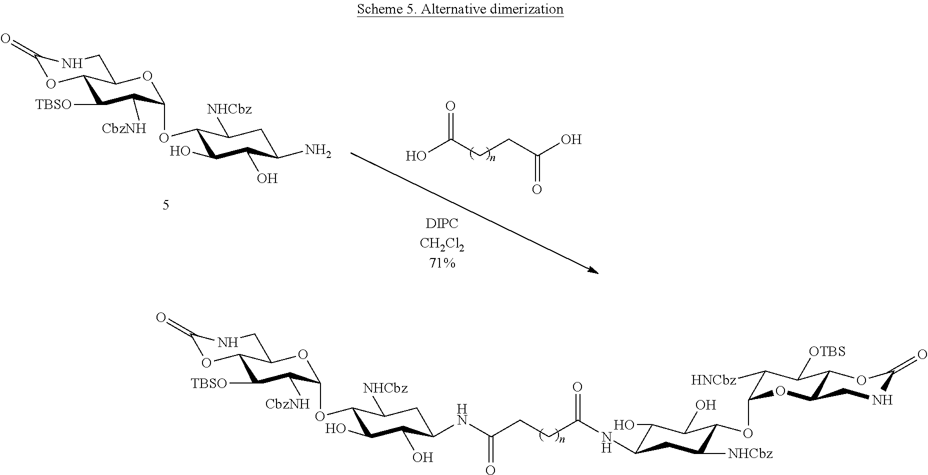

Desilylation also proved cumbersome on such highly functionalized molecules, and only acidic treatment with hydrogen chloride in methanol provided an efficient and clean reaction. Both silyl groups were indeed removed in these conditions leading to 10 in quantitative yield. Finally, the two remaining 6-membered cyclic carbamates were removed by decarboxylation upon treatment with barium hydroxide in carefully controlled conditions, leading to the hexaamine 11. After column chromatography, the pure product was protonated by treatment with 1M aqueous hydrochloric acid in order to recover a good water solubility for biological tests and reliable NMR analysis. The corresponding hexachlorhydrate salt 12 was obtained with an overall yield of 10% over 9 steps from neamine 1.

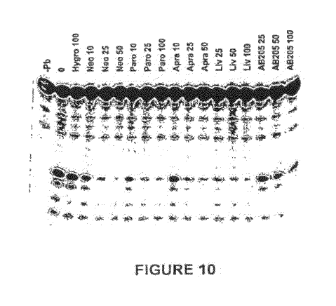

Preliminary structural and biological studies revealed that this dimer 12 binds as expected to the DIS ‘kissing complex’. Biochemical studies relying on a selective Pb²⁺ induced cleavage of the DIS loop (Vicens et al., Structure 2001, 9:647-658; Francois, et al., Nucleic Acids Res. 2005, 33:5677-5690; Paillard et al., J. Mol. Biol. 1997, 270:36-49) showed that the dimer 12 binds to DIS. It indeed protected DIS from Pb²⁺ cleavage (FIG. 10), although its effect seemed slightly less efficient than neomycin and paromomycin in the standard conditions used.

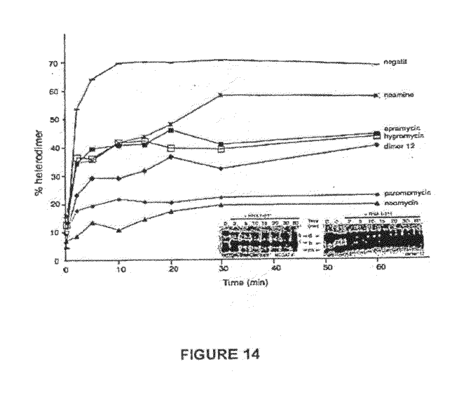

Dimer 12 stabilizes the kissing complex at 37° C. in the context of 615 nucleotide RNA fragments. Competition experiments between long and short RNA fragments (615 and 311 nucleotides, respectively) were performed in the presence or not of aminoglycoside and dimer 12, the binding of which should slow down the heterodimer formation (FIG. 14). While dimer 12 allows the formation of less than 40% heterodimer (RNA615/RNA311), twice less heterodimer is formed with neomycin, lividomycin and paromomycin. However, all other aminoglycosides tested in this study seemed less efficient than dimer 12 (FIG. 14). These results suggest that dimer 12 is able to bind to the DIS and to reduce the dissociation of the kissing complex.

Without limiting the scope the present invention, the following schemes produce dimers representing compounds contemplated by the present invention:

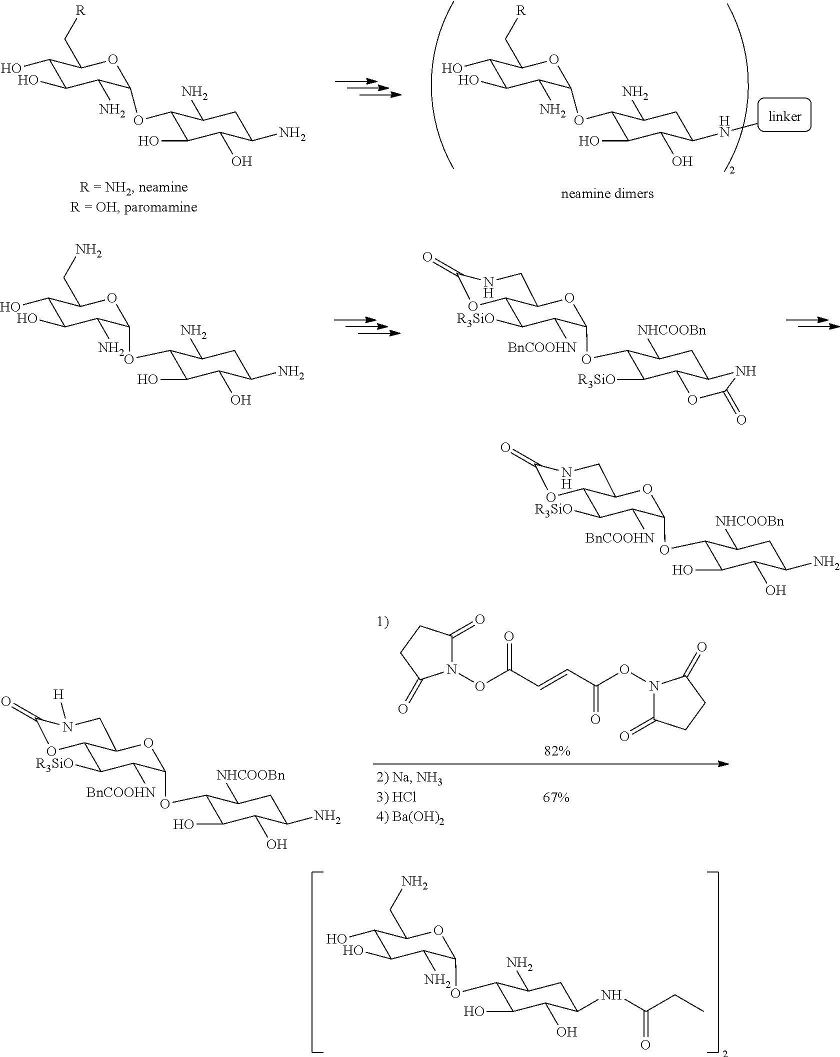

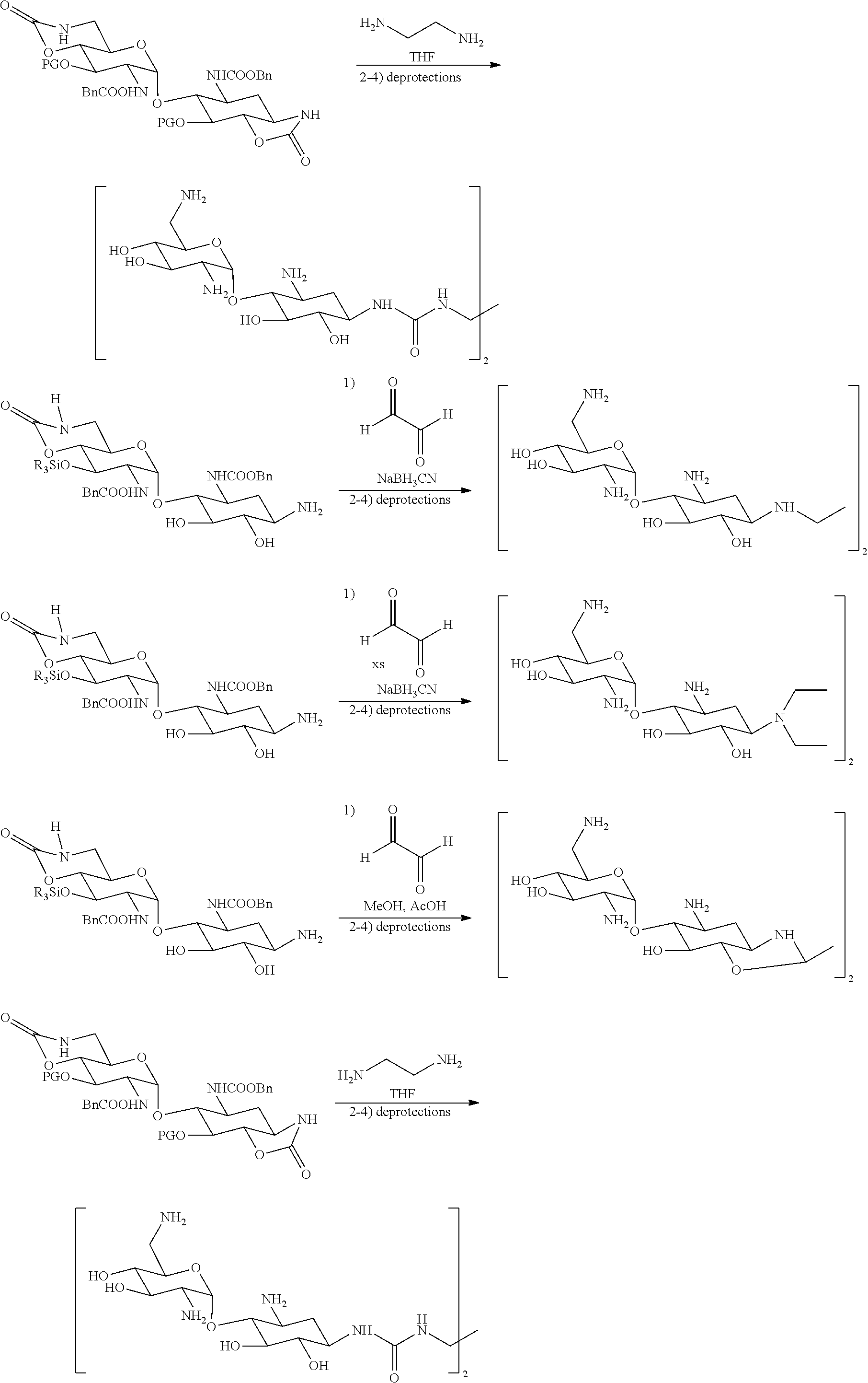

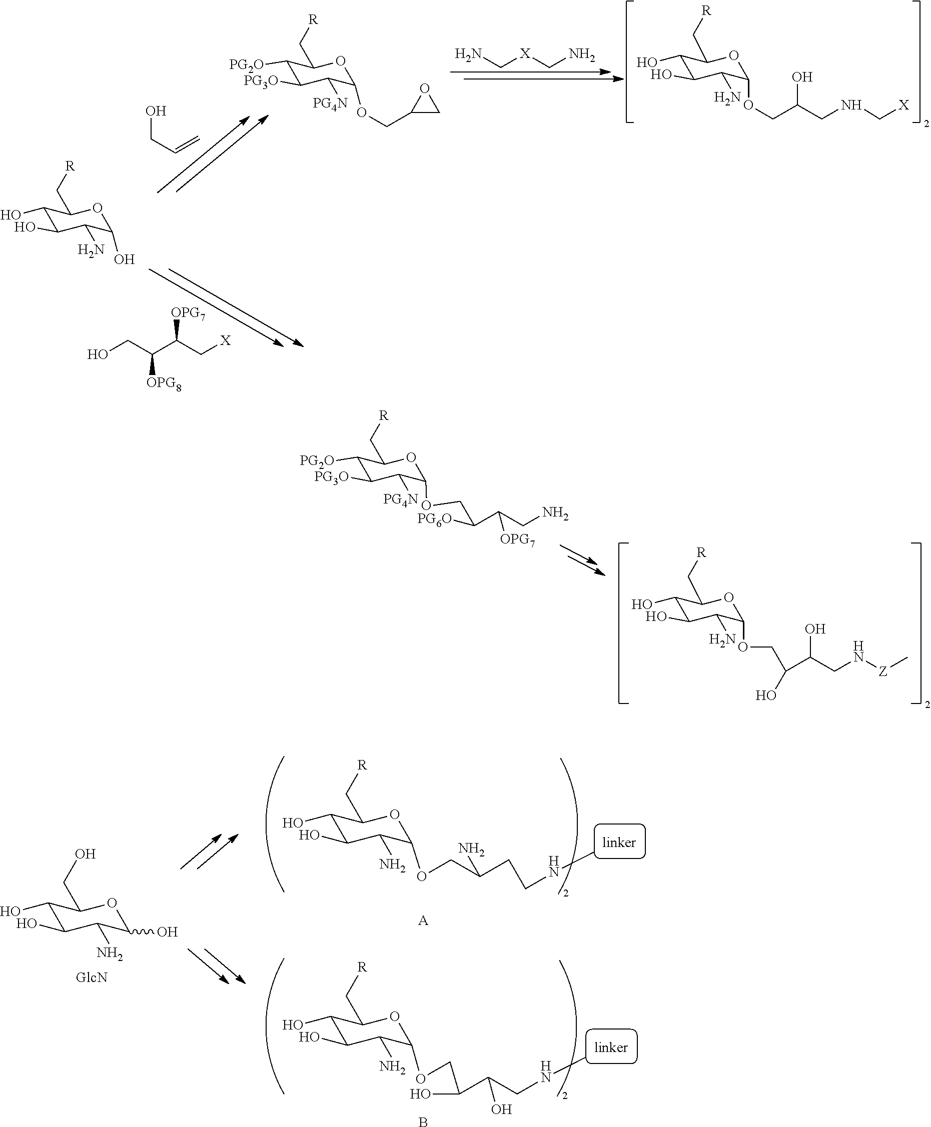

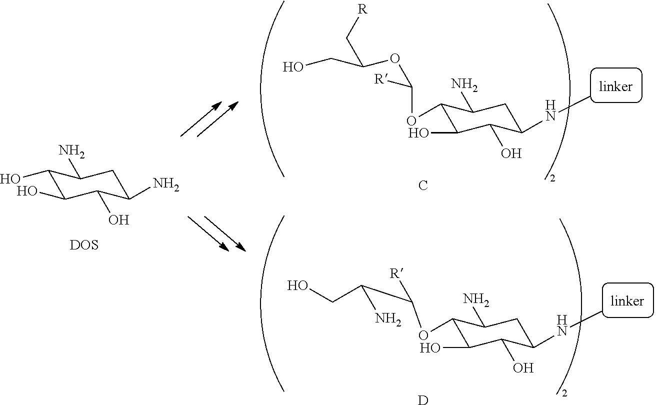

Further, and without limiting the scope the present invention, the following compounds are contemplated by the present invention:

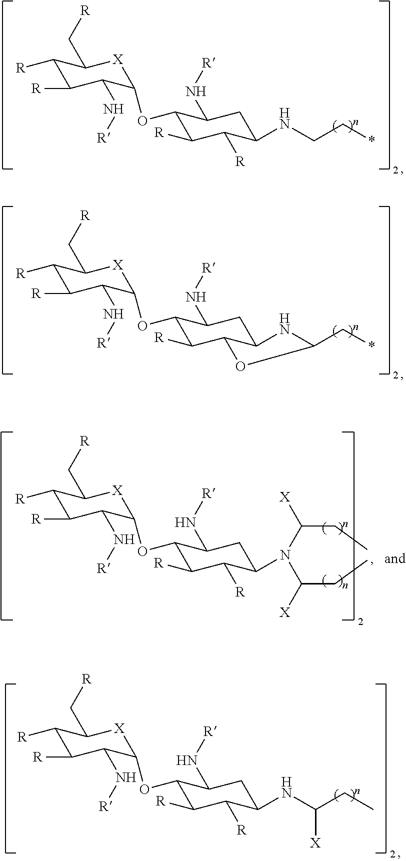

wherein R is selected from an amine or a hydroxyl group or an alkyl chain containing a hydroxyl or amine, R′ is selected from a hydrogen, oxygen or nitrogen atom or a straight or branched, saturated or unsaturated, alkyl group of between 1 and 10 carbons, X is selected from an oxygen atom, hydroxyl, nitrogen atom, amine, sulfur atom or a carbon atom and n is 1 to 15, or its pharmaceutically acceptable salt or ester.

### Administration

Humans infected with HIV can be treated by the inhalation, systemic, oral, topical, or transdermal administration of a composition comprising an effective amount of the compounds described herein or a pharmaceutically acceptable salt, ester or prodrug thereof, optionally in a pharmaceutically acceptable carrier, diluent or excipient.

In accordance with the present invention, the anti-HIV-1 drug of the present invention may be mixed with one or more pharmaceutical agents (i.e., carriers, diluents, or excipients) into a single formulation. In addition, the anti-HIV-1 drug of the present invention may be mixed with one or more antibiotics or therapeutic agent. The molecules and the agents may also be formulated and delivered separately.

The compositions described herein can be provided as physiologically acceptable formulations using known techniques, and the formulations can be administered by standard routes. In general, pharmaceutical composition can be administered by topical, oral, rectal or parenteral (e.g., intravenous, subcutaneous or intramuscular) route. In addition, the compositions can be incorporated into polymers allowing for sustained release, the polymers being implanted in the vicinity of where delivery is desired, or the polymers can be implanted, for example, subcutaneously or intramuscularly or delivered intravenously or intraperitoneally to result in systemic delivery of the anti-HIV-1 drug. Other formulations for controlled, prolonged release of therapeutic agents useful in the present invention are disclosed in U.S. Pat. No. 6,706,289, the disclosure of which is incorporated herein by reference.

A sustained release matrix, as used herein, is a matrix made of materials, usually polymers which are degradable by enzymatic or acid/base hydrolysis or by dissolution. Once inserted into the body, the matrix is acted upon by enzymes and body fluids. The sustained release matrix desirably is chosen by biocompatible materials including, but not limited to, liposomes, polylactides (polylactide acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such phenylalanine, tyrosine, isoleucine, polynucleotides, polyvinyl propylene, polyvinylpyrrolidone and silicone.

The dosage of the composition will depend on the particular composition used, and other clinical factors such as weight and condition of the patient, and the route of administration.

Further, the term “effective amount” refers to the amount of the composition which, when administered to a human or animal, targets the DIS of HIV-1 RNA thereby inhibiting or preventing the replication of HIV-1 RNA. The effective amount is readily determined by one of skill in the art following routine procedures.

For example, compositions of the present invention may be administered parenterally or orally in a range of approximately 1.0 μg to 1.0 g per dose, though this range is not intended to be limiting. The actual amount of composition required to elicit an appropriate response will vary for each individual patient depending on the potency of the composition administered and on the response of the individual. Consequently, the specific amount administered to an individual will be determined by routine experimentation and based upon the training treatment of diseases. For example, it is contemplated that the anti-HIV-1 drug would be administered daily with the dosage in the range of from 0.1 to 500 mg/kg/day.

The formulations in accordance with the present invention can be administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

The formulations include those suitable for oral, rectal, nasal, inhalation, topical (including dermal, transdermal, buccal and sublingual), vaginal, parenteral (including subcutaneous, intramuscular, intravenous, intradermal, intraocular, intratracheal, and epidural) or inhalation administration. The formulations can conveniently be presented in unit dosage form and can be prepared by conventional pharmaceutical techniques. Such techniques include the step of bringing into association the active ingredient and a pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion, etc.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface-active or dispersing agent. Molded tablets may be made by molding, in a suitable machine, a mixture of the powdered compound moistened with an inert liquid diluent. The tablets may optionally be coated or scored and may be formulated so as to provide a slow or controlled release of the active ingredient therein.

Formulations suitable for topical administration in the mouth include lozenges comprising the ingredients in a flavored base, usually sucrose and acacia or tragacanth; pastilles comprising the active ingredient in an inert base such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. In one embodiment the topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is taken; i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing, in addition to the active ingredient, ingredients such as carriers as are known in the art to be appropriate.

Formulation suitable for inhalation may be presented as mists, dusts, powders or spray formulations containing, in addition to the active ingredient, ingredients such as carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Formulations suitable for parenteral administration also include, but are not limited to, nanoparticle formulations made by numerous methods as disclosed in U.S. patent application Ser. No. 10/392,403 (Publication No. US 2004/0033267), U.S. patent application Ser. No. 10/412,669 (Publication No. US 2003/0219490), U.S. Pat. No. 5,494,683, U.S. patent application Ser. No. 10/878,623 (Publication No. US 2005/0008707), U.S. Pat. No. 5,510,118, U.S. Pat. No. 5,524,270, U.S. Pat. No. 5,145,684, U.S. Pat. No. 5,399,363, U.S. Pat. No. 5,518,187, U.S. Pat. No. 5,862,999, U.S. Pat. No. 5,718,388, and U.S. Pat. No. 6,267,989, all of which are hereby incorporated herein by reference in there entirety. A review of drug formulation technology is provided in “Water Insoluble Drug Formulation” by Rong Liu, editor, 2000, pp. 1-633, CRC Press LLC, which is incorporated herein by reference in its entirety.

By forming nanoparticles, the compositions disclosed herein may have increased bioavailability. Preferably, the particles are comprised of the anti-HIV-1 drug, alone or in combination, with accessory ingredients or in a polymer for sustained release. The particles of the compounds of the present invention have an effective average particle size of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 run, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods well known to those of ordinary skill in the art. It is understood that the particle sizes are average particle sizes and the actual particle sizes will vary in any particular formulation. Often, surface stabilizers are used to form stable nanoparticles; however, this method of forming nanoparticles is only one of many different methods of forming effective nanoparticle compositions. The formulations may be presented in unit-dose or multi-dose containers, for example, sealed ampules and vials, and may be stored in freeze-dried (lyophilized) conditions requiring only the addition of a sterile liquid carrier, for example, water for injections, immediately prior to use. Extemporaneous injection solutions and suspensions may be prepared from sterile powders, granules and tablets of the kinds previously described.

In one embodiment, the anti-HIV-1 drug and pharmaceutical agent can be administered simultaneously. In another embodiment, they can be administered separately. Mixtures of more than pharmaceutical agent can, of course, be administered. Indeed, it is often desirable to use mixtures or sequential administrations of different pharmaceutical agents, especially pharmaceutical agents from different classes.

If the anti-HIV-1 drug formulation and the pharmaceutical agent are to be administered sequentially, the amount of time between administration of the anti-HIV-1 drug formulation and the pharmaceutical agent will depend upon factors such as the amount of time it take the anti-HIV-1 drug formulation to be fully incorporated into the circulatory system of the host and the retention time of the anti-HIV-1 drug formulation in the host's body.

The anti-HIV-1 drug is administered in a therapeutically effective amount. This amount will be determined on an individual basis and will be based, at least in part, on consideration of the host's size, the severity of the viral infection to be treated, the results sought, and other such considerations. An effective amount can be determined by one of ordinary skill in the art employing such factors and using no more than routine experimentation.

It should be understood that, in addition to the ingredients particularly mentioned above, the formulations of the present invention may include other agents conventional in the art having regard to the type of formulation in question, for example, those suitable for oral administration may include flavoring agents, and nanoparticle formulations (e.g.; less than 2000 nanometers, preferably less than 1000 nanometers, most preferably less than 500 nanometers in average cross section) may include one or more than one excipient chosen to prevent particle agglomeration.

### Pharmaceutical Preparations

Also contemplated by the present invention are implants or other devices comprised of the formulation in accordance with the disclosed embodiments, or prodrugs thereof, or other compounds included by reference where the drug or prodrug is formulated in a biodegradable or non-biodegradable polymer for sustained release. Non-biodegradable polymers release the drug in a controlled fashion through physical or mechanical processes without the polymer itself being degraded. Biodegradable polymers are designed to gradually be hydrolyzed or solubilized by natural processes in the body, allowing gradual release of the admixed drug or prodrug. The drug or prodrug can be chemically linked to the polymer or can be incorporated into the polymer by admixture. Both biodegradable and non-biodegradable polymers and the process by which drugs are incorporated into the polymers for controlled release are well known to those skilled in the art. Examples of such polymers can be found in many references well know to those of ordinary skill in the art, such as Biodegradable Polymers as Drug Delivery Systems, M. Chasin, R. Langer (editor), Marcel Dekker, Inc. 1990. These implants or devices can be implanted in the vicinity where delivery is desired, or can be introduced so as to result in systemic delivery of the agent.

Any of the compounds described herein for combination or alternative therapy can be administered as any prodrug that, upon administration to the recipient, is capable of providing, directly or indirectly, the parent compound.

A person skilled in the art will be able by reference to standard texts, such as Remington's Pharmaceutical Sciences 17th edition, to determine how the formulations are to be made and how these may be administered.

In a still further aspect of the present invention there is provided a method of prophylaxis or treatment of a viral infection, said method including administering to a patient in need of such prophylaxis or treatment an effective amount of compounds of the anti-HIV-1 drug, or prodrugs thereof, in combination with a pharmaceutical agent according to the disclosed embodiment, as described herein. It should be understood that prophylaxis or treatment of said condition includes amelioration of said condition.

Pharmaceutically acceptable salts of the compounds, or the prodrugs thereof, can be prepared in any conventional manner, for example from the free base and acid. In vivo hydrolysable esters, amides and carbamates and other acceptable prodrugs can be prepared in any conventional manner.

100% pure isomers are contemplated by this invention; however a stereochemical isomer (labeled as a or 3, or as R or S) may be a mixture of both in any ratio, where it is chemically possible by one skilled in the art. Also contemplated by this invention are both classical and non-classical bioisosteric atom and substituent replacements, such as are described by Patani and Lavoie (“Bio-isosterism: a rational approach in drug design” Chem. Rev. 1996, pp. 3147-3176) and are well known to one skilled in the art. Such bioisosteric replacements include, for example, but are not limited to, substitution of ═S or ═NH for ═O.

Known compounds that are used in accordance with the invention and precursors to novel compounds according to the invention can be purchased commercially. Other compounds according to the invention can be synthesized according to known methods from publicly available precursors.

The compositions and methods are further illustrated by the following non-limiting examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention.

### EXAMPLE 1

### Crystallization of the DIS/Aminoglycoside Complexes

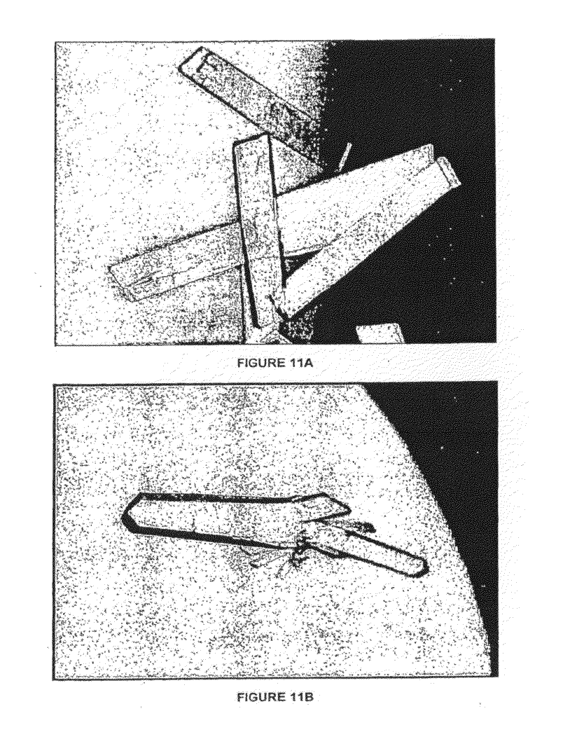

Co-crystals with neamine, ribostamycin, neomycin, lividomycin and neamine dimer have been obtained (FIGS. 11A and 11B) and the structures of four DIS complexes were solved at 1.8- to 2.2 Å resolution. These structures are very similar and mainly differ by the size of the ligand. In all complexes, two aminoglycosides interact with the two (A-site)-like motifs of the loop-loop complex.

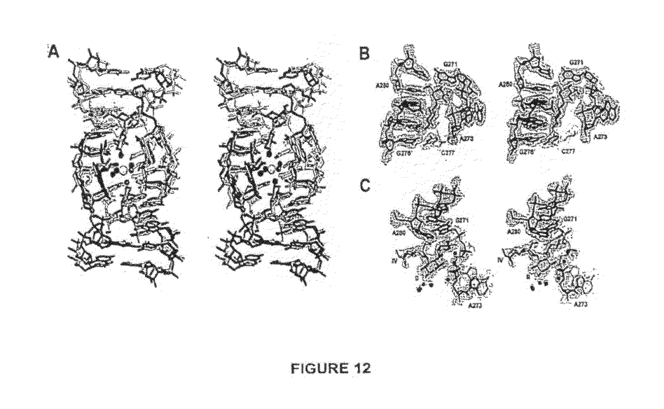

Analysis of the contacts between the aminoglycoside and the DIS reveals that, as for the A site, rings I, II and III are responsible for the sequence and structure specificity of aminoglycoside binding (FIG. 12). The structure of a neamine dimer-DIS complex (FIG. 11B) has been resolved at low level; however, it clearly showed the presence of the molecule at the right place. This result validates the overall concept.

The flipped out conformation of the first two purines of the DIS loop was confirmed and more interestingly, several contacts are specific for the kissing-loop complex, and some aminoglycosides build more direct interactions with the kissing-loop complex than with the A site (FIG. 12).

Materials and Methods: A 23-mer RNA corresponding to the subtype F DIS was purchased from Dharmacon (Boulder, Colo., USA) and purified as described (Ennifar et al., Nucleic Acids Res. 2003, 31:2671-2682). For structure solution, a 5-bromo-uridine was substituted for uridine 3 of the 23-mer RNA. RNA was heated in water at 90° C. for 3 min and chilled on ice for 10 min. Buffer was then added to reach a final concentration of 150 mM KCl, 5 mM MgCl₂ and 20 mM sodium cacodylate pH 7.0. RNA was then concentrated on a Centricon 10K (Millipore) to a final concentration of 300-360 μM. All aminoglycosides were obtained from Sigma and used without purification, except neamine, which was obtained from neomycin by acidic treatment. Crystals of aminoglycoside/DIS complexes were obtained by mixing 7 μl of the RNA solution with 1.8 μl of a solution containing 5 mM neamine chloride or ribostamycin sulfate in 30% (w/v) 2,4-methylpentanediol (MPD) or with 1.0 μl of a solution containing 5 mM neomycin sulfate or 5 mM lividomycin sulfate in 30% MPD. Sitting drops were equilibrated over two days at 37° C. against a reservoir containing 40% MPD, 300 mM KCl, 50 mM sodium cacodylate pH 7.0, 20 mM MgCl₂ and crystals were transferred at 20° C. for stabilization. Crystals were frozen in liquid ethane prior to data collection.

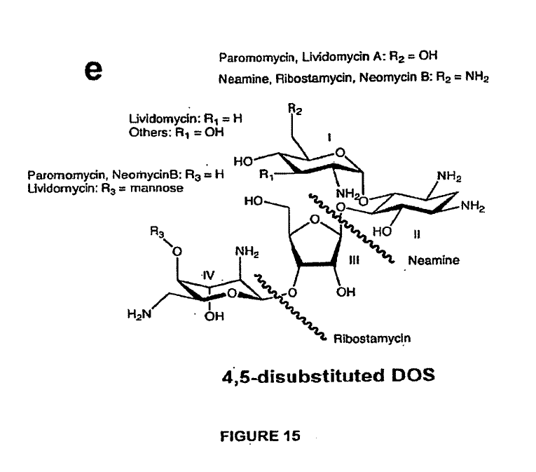

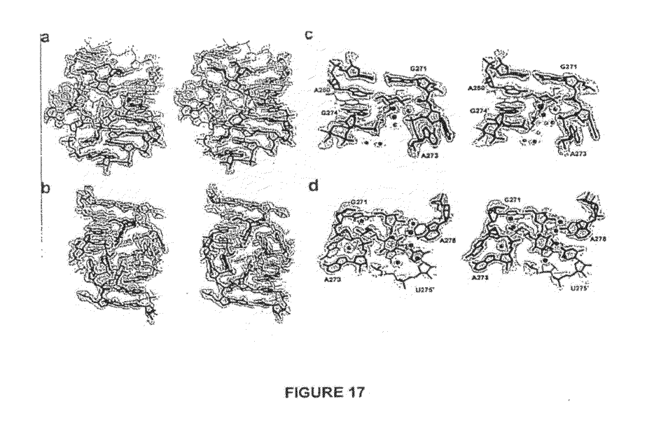

Results: Co-crystallization of a 23-mer RNA corresponding to the subtype F DIS with several 4,5-disubstituted and 4,6-disubstituted DOS derivatives was performed (FIGS. 14 and 15). Co-crystals with neamine, ribostamycin, neomycin and lividomycin, and the structures of the four DIS/4,5-disubstituted DOS complexes were solved at 1.8-to 2.2 Å resolution (Table 1). These structures are very similar and mainly differ by the size of the ligand. In all complexes, two aminoglycosides interact with the two (A-site)-like motifs of the loop-loop complex (FIGS. 17a and 17b). The average distance separating the two aminoglycosides is 4.4 Å. RNA conformational change following ligand binding is restricted to a ribose pucker shift of G₂₇₁ from C2′-endo in most free structures (Ennifar et al., Nat. Struct. Biol. 2001, 8:1064-1068) to C3′-endo conformation in present structures (FIG. 12). This movement induced an opening of the aminoglycoside pocket, avoiding a steric conflict between cycle I of the 4,5-disubstituted DOS and G₂₇₁ phosphate and ribose.

The structure of an unliganded form of the subtype F kissing-loop complex in which the ribose pucker of G₂₇₁ is C3′-endo was solved (Ennifar et al., J. Mol. Biol. 2006, 356: 771-782). This structure is also characterized by a continuous stacking of the four bulged out purines (two in each RNA molecule) of the kissing-loop complex, contrasting with the two pairs of stacked purines separated by a gap observed in most DIS kissing-loop (Ennifar et al., J. Mol. Biol. 2006, 356: 771-782). Superimposition of the structures shows that binding of 4,5-disubstituted DOS to this conformation of the kissing-loop complex also requires a structural rearrangement of the binding pocket to prevent a steric clash with phosphate 272, suggesting that aminoglycoside binding is incompatible with continuous stacking of the four bulged out purines.

Aminoglycoside binding in the DIS pocket results in the displacement of the hexahydrated magnesium, bound to the 5′UGCA3′ sequence of the intermolecular helix of the unliganded kissing-loop complex, that is required for dimerization of non-subtype B DIS. This displacement is obligatory because the two axial water molecules bound to O4 of U₂₇₅ and to the magnesium are replaced by the two positively charged N1 amino groups of each DOS ring (FIG. 12). This likely explains why magnesium is dispensable for DIS dimerization in presence of aminoglycosides.

Each aminoglycoside molecule interacts simultaneously with both RNA molecules of the loop-loop helix. Interactions are either direct, or mediated by conserved water molecules or cations (FIGS. 12, 17c, 17d, and 18). Ring II (the DOS ring) almost exclusively interacts with one RNA molecule (FIGS. 17c, 17d & 18), while rings I, III and IV bind only to the other RNA molecule (FIGS. 17c, 17d & 18). A total of 15 direct antibiotic-RNA contacts are observed for the neomycin-DIS complex (FIG. 18), i.e. more than in the equivalent paromomycin-ribosomal A site complex, which is stabilized by 11 direct contacts). Analysis of these contacts reveals that, as for the A site, rings I, II and III are responsible for the sequence and structure specificity of aminoglycoside binding: base-specific contacts involve residues G₂₇₁, A₂₇₈, C₂₇₉, G₂₇₄^{.} and U_{275′}, as well as A₂₈₀ which forms a Watson-Crick-like base pair with ring I (FIG. 18). Many of these contacts were also observed between this “ribostamycin core” and the ribosomal A site. Some contacts are specific for the DIS/4,5-disubstituted DOS complexes: e.g. with the A₂₇₈ of the U₂₇₅-A_{278′}. base-pair replacing U₁₄₀₆ in the universal U_{1406-U}₁₄₉₅ mismatch in the ribosomal 18S RNA. Two phosphates of the bulged-out A₁₄₉₂ and A₁₄₉₃ of the ribosomal RNA make direct contacts with O3′ and O4′ of ring I (Carter et al., Nature 2000, 407:340-348; Vicens et al., Structure 2001, 9:647-658). Because of the difference in topology between the two RNA structures (loop-loop complex vs duplex), these phosphates are displaced in the DIS compared to the ribosomal A site (FIGS. 19A and 19B). In spite of this difference, ring I is able to strongly bind to the loop-loop backbone owing to four direct interactions involving O4′, O3′ and N2′ with the phosphate moiety of A₂₇₂ and A₂₇₃ (FIGS. 17c, 17d & 18). In addition, the high quality of the electron density maps allowed the observation of a complex network of well-defined water molecules reinforcing the drug-RNA interaction. Water bridges were also observed between the two aminoglycoside molecules within a loop-loop complex (FIGS. 12A and 18). A conserved potassium cation was observed at the drug-RNA interface, where it mediates contacts between ring I and the phosphate group of A₂₇₂ and A₂₇₃ (FIGS. 12A, 17c, 17d, and 18). This peculiar mode of potassium binding is present neither in the unliganded DIS loop-loop complex, nor in the aminoglycoside/ribosomal A site complexes. Thus, this is a specific feature of the DIS-aminoglycoside complexes. As the O3′ hydroxyl group is lacking in lividomycin (FIG. 15), the potassium ion is not present in the DIS/lividomycin complex (FIG. 12), and direct contacts involving this position are also lost.

At variance with the “ribostamycin core”, rings IV and V bind more loosely to the DIS and are exclusively involved into non-specific interactions with the RNA backbone. These rings adopt several conformations in the RNA deep groove, and they are characterized by high temperature factors, contrasting with the very low temperature factors of rings I and II and the surrounding RNA. As a consequence, the density for ring V, and also to some extent for ring IV, is poor (FIG. 12), as also observed in the complexes with the ribosomal A site (Francois et al., Nucleic Acids Res. 2005, 33:5677-5690; Vicens et al., Structure 2001, 9:647-658). This flexibility is confirmed by a superimposition of rings I and II of paromomycin bound to the ribosomal A site (Vicens et al., Structure 2001, 9:647-658) to the aminoglycosides observed in the present structures (FIGS. 19A and 19B). Rings I, II and III adopt a unique conformation in all structures, whereas rings IV and V (by comparing the two molecules in the asymmetric unit) can adopt different orientations.

### EXAMPLE 2

### X-Ray Collection, Structure Solution and Refinement

Materials and Methods: Data were collected at 100 or 120 K on ID23-1 and ID-29 at the ESRF (Grenoble, France) or on X06SA at the SLS (Villigen, Switzerland) (Table 1) and processed with the HKL package (Otwinowski et al., In Methods in Enzymology, J. C. W. Carter, and R. M. Sweet, eds. (Academic Press), 1996, pp. 307-326.). The two bromide sites of the neamine/DIS complex were located with CNS (Brunger et al., Acta Crystallogr. D. Biol. Crystallogr. 1998, 54:905-921) using the anomalous signal. The structure was solved with CNS by MAD using a three wavelength experiment, followed by solvent flattening with 50% solvent content. This structure was then used to solve the ribostamycin/DIS complex by molecular replacement using CNS with PC refinement, and the neomycin/DIS complex using Molrep (Vagin et al., Acta Crystallogr. D. Biol. Crystallogr. 2001 57:1451-1456) with advanced rotation function and translation function options. The lividomycin/DIS complex was solved by rigid body refinement of the neomycin/DIS complex. Molecular replacement attempts using unbound DIS kissing-loop complexes all remained unsuccessful. All structures were refined with CNS. Potassium sites were localized by anomalous difference maps using data sets of the neomycin-DIS and ribostamycin-DIS complexes collected at 1.5 or 1.65 Å wavelengths to maximize the anomalous signal of the potassium (f″=1.01 ē). Occupancy of bromide in bromo-uridines was not set to 1.0 and refined due to strong radiolysis during data collection (Ennifar et al., Acta Crystallogr. D. Biol. Crystallogr. 2002, 58:1262-1268).

TABLE 1
Data collected and refinement statistics

| | Neamine^{a} | Ribostamycin | Neomycin | Lividomycin^{a} |
|---|---|---|---|---|
| PBD ID | 2FCX | 2FCZ | 2FCY | 2FD0 |
| Space group | C222₁ | C2 | C222₁ | C222₁ |
| a (Å) | 27.0 | 112.1 | 27.2 | 27.1 |
| b (Å) | 113.4 | 27.2 | 117.8 | 115.3 |
| c (Å) | 95.0 | 106.9 | 94.6 | 95.3 |
| β | 90° | 116.7° | 90° | 90° |
| Beamline | X06SA | X06SA | X06SA | X06SA |
| Wavelength (Å) | 0.91961 | 0.91946 | 0.91946 | 0.91946 |
| Resolution range (Å) | 40-2.0 | 20-2.0 | 40-2.2 | 40-1.8 |
| Average redundancy | 6.8 | 3.3 | 4.3 | 6.3 |
| Unique flexions | 18 852 | 18 764 | 7689 | 26 564 |
| Completeness^{b} | 98.6 (92.7) | 95.4 (99.2) | 96.7 (90.2) | 99.3 (98.7) |
| Rsym^{b} | 7.4 (27.3) | 6.3 (25.6) | 5.9 (23.1) | 6.1 (27.3) |
| Average I/σ^{b} | 24.4 (4.0) | 18.9 (5.7) | 20.7 (6.4) | 26.4 (3.5) |
| R/Rfree | 26.7/29.2 | 23.8/25.6 | 23.5/25.3 | 24.5/24.5 |
| Water molecules | 35 | 208 | 74 | 90 |
| Cations | 3 K⁺ | 4 K⁺ | 6 K⁺ | 4 K⁺ |
| Anions | 1 Cl⁻ | 0 | 1 Cl⁻, 1 SO₄²⁻ | 1 Cl⁻ |
| Aminoglyosides | 2 | 4 | 2 | 2 |

^{a}Friedel mates were processed separately
^{b}Values for last resolution shell are shown in parenthesis

### EXAMPLE 3

### In vitro Footprinting of Aminoglycosides

Materials and Methods: Chemical footprinting experiments were performed on a 23-mer subtype A SL1 RNA or on RNA1-615, which corresponds to nucleotides 1-615 of HIV-1 Mal genomic RNA (Ennifar et al., J. Biol. Chem. 2003 278:2723-2730). Chemical modification was carried out either with dimethyl sulfate (DMS, Fluka) to test the accessibility of N1-A and N3-C positions or with lead acetate (Merck), which selectivity cleaves the subtype A DIS loop between the first and second nucleotides.

### EXAMPLE 4

### Infectious HIV-1 Molecular Clones, Cell Culture. Transfections and Infections

Materials and Methods: The HIV-1 NL4.3 molecular clone was used to generate mutant constructs with nucleotides 272-280 (DIS loop) from the subtype A and F isolates substituted for the homologous NL4.3 region. To obtain these constructs, the QuickChange™ site-directed mutagenesis kit was used according to the manufacturer (Stratagene), using plasmid DNA pLTR5′-NL4.3 (Paillart et al., J. Virol. 1996, 70:8348-8354). The resulting mutant plasmids were digested with AatII and SphI and the AatII-SphI fragment was substituted for the homologous region of pNL4.3. Mutations were confirmed by DNA sequencing.

HeLa and H9 cell cultures and transfections were performed as previously described (Goldschmidt et al., J. Biol. Chem, 2004, 279:35923-35931). Viral replication of wild-type and mutant viruses was monitored by measuring RT activity in the culture supernatant of infected H9 cells.

### EXAMPLE 5

### In vitro Binding of Aminoglycoside Antibiotics to the DIS

The binding of aminoglycosides to DIS can be detected through several biochemical methods: it induces protection toward chemical modification with dimethyl sulfate (DMS) or toward a specific cleavage in the subtype A promoted by lead (II); it also induces slower migration on non-denaturated gel.

As expected, paromomycin and neomycin protect from DMS modification the bases implicated in the interaction for the Mal sequence, whereas there is no significant interaction of the same antibiotics for the NL4.3 sequence. In the subtype A, neomycin protects from DMS modification the loop adenine A280 and the guanine G274, but no protection is observed with tobramycin. The dimeric molecules also protect from DMS modification as neomycin (FIG. 10).

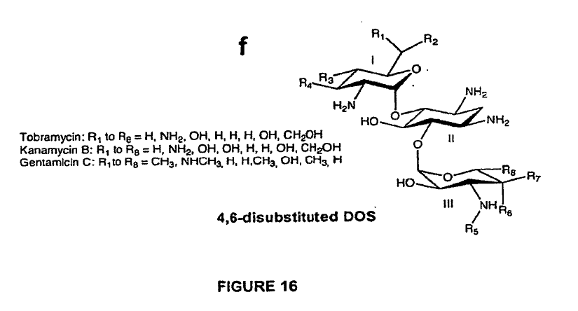

Results: Even though a number of 4,5-disubstituted and 4,6-disubstituted DOS derivatives were tested, only co-crystals of the DIS kissing-loop complex with 4,5-disubstituted compounds were obtained. Attempts at co-crystallizing 23-mer SL1 RNA with tobramycin or kanamycin B, two 4,6-disubstituted DOS (FIG. 16), resulted in crystals containing only the RNA. Modeling of 4,6-disubstituted compounds in the kissing-loop complex based on the structural analogy with the ribosomal A site and the structure of this site with tobramycin and gentamicin, suggests that these compounds would clash with positions N4 of C₂₇₇ and N6 of A₂₇₈ in the kissing-loop complex, where it differs from the ribosomal A site (FIG. 20). However, minor changes in the RNA structure would be sufficient to allow binding of 4,6-disubstituted DOS to the kissing-loop complex.

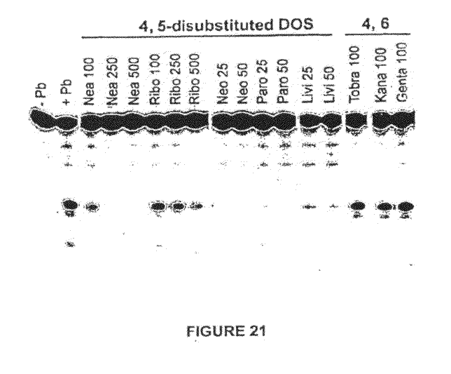

At this stage, it was unclear if this class of compound is able to bind to the DIS kissing-loop complex. To compare binding of 4,5-disubstituted and 4,6-disubstituted DOS, a 23-mer RNA corresponding to the subtype A DIS, which was allowed to dimerize in conditions permitting crystallization of the kissing-loop complex, was used. As previously shown, lead (II) induced a specific cleavage in the subtype A DIS loop that is sensitive to the binding of aminoglycoside (FIG. 21). Remarkably, none of the 4,6-disubstituted DOS tested inhibited this cleavage at 100 μM, suggesting that they did not bind to the kissing-loop complex. Among the 4,5-disubstituted DOS that co-crystallized with the subtype F kissing-loop complex, neomycin and lividomycin completely inhibited lead-induced cleavage of the subtype A DIS loop at 25 μM, whereas neamine and ribostamycin were less efficient. Unexpectedly, the bicyclic neamine inhibited cleavage at lower concentration than the tricyclic ribostamycin (FIG. 21). Neamine has also a higher affinity than ribostamycin for the ribosomal A site (Wong et al., Chem. Biol. 1998, 5:397-406), but neither the crystal structures of these aminoglycosides with the DIS kissing-loop complex as disclosed herein nor those with the ribosomal A site (Francois et al., Nucleic Acids Res. 2005 33:5677-5690) provide a clear explanation for these observations. Neomycin and paromomycin, which have very similar chemical structures, equally inhibited the lead-induced cleavage of the DIS loop.

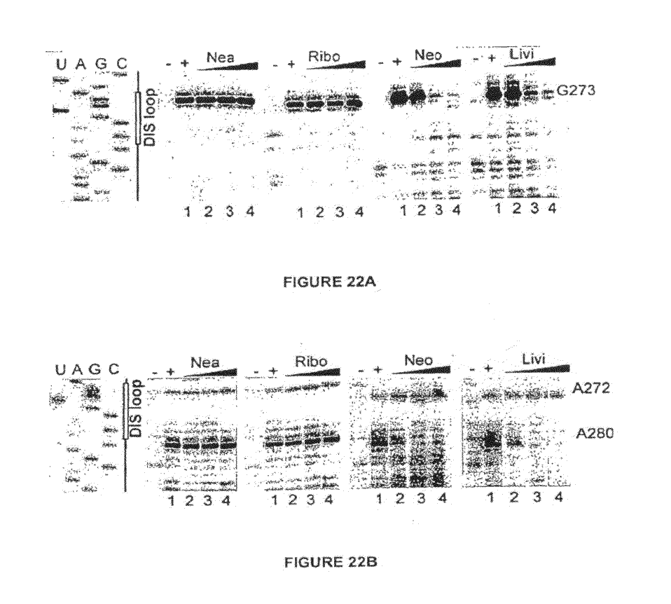

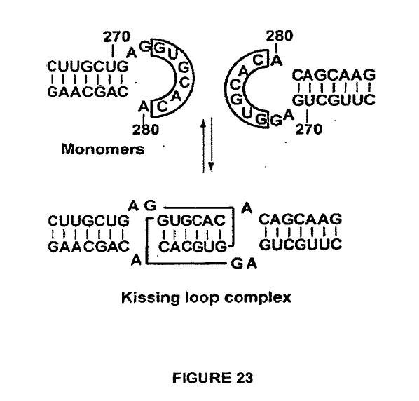

To evaluate the capacity of 4,5-disubstituted DOS to bind to the DIS kissing-loop complex in a larger RNA presenting numerous unspecific binding sites, RNA1-615, which corresponds to nucleotides 1 to 615 of the HIV-1 MAL genomic RNA, was used. This recombinant isolate possesses a subtype A DIS. Using a primer extension assay, the lead-induced cleavage between nucleotides A₂₇₂ and G₂₇₃ (in this isolate, the DIS loop corresponds to nucleotides 272 to 280; see FIG. 23) was almost completely inhibited at 50 μM neomycin and lividomycin (FIG. 22A). Similar results were obtained with paromomycin. However, no inhibition was observed with neamine and ribostamycin, even at 100 μM (FIG. 22A).

To confirm these results, DMS footprinting was performed (FIG. 22B). Residues A₂₇₂ and A₂₈₀ of RNA1-615 are unpaired in the kissing-loop complex and are methylated by DMS at their N1 position. As previously observed (Baudin et al., J. Mol. Biol. 1993, 229:382-397; Paillart et al., J. Mol. Biol. 1997, 270:36-49), A₂₈₀ is more reactive than A₂₇₂, even though the latter nucleotide is flipped out of the helix, while the former is stacked inside the structure. At 10 μM and above, neomycin and lividomycin strongly protected A₂₈₀ against methylation (FIG. 22B). This result was predictable in light of the pseudo-Watson-Crick interaction between this base and ring I of 4,5-disubstituted DOS (FIG. 18). On the other hand, A₂₇₂, which is flipped out of the kissing-loop complex was not protected against methylation by DMS (FIG. 22B). As for the lead-induced cleavage, neamine and ribostamycin provided no protection against methylation, even at 100 μM (FIG. 22B). Thus, even though these compounds bound to short DIS RNA in the same way as neomycin and lividomycin (FIGS. 19A and 19B), they were unable to efficiently recognize this target in the context of a large RNA.

Using DMS footprinting, it was discovered that neomycin, paromomycin and lividomycin also efficiently bound to the subtype F kissing-loop complex in the context of RNA1-615, while neamine and ribostamycin did not bind. In addition, none of the 4,5-disubstituted DOS was bound to the subtype B kissing-loop complex.

### EXAMPLE 6

### Stabilization of the Kissing-Loop Complex by 4,5-Disubstituted DOS

Materials and Methods: In a typical experiment, unlabeled 1-615 RNA (400 nM), together with a corresponding internally labeled RNA (3-5 nCi), were heated for 2 min at 90° C., chilled for 2 min on ice, and dimerization was initiated by addition of 2 μl of 5-fold concentrated dimerization buffer (final concentration: 50 mM sodium cacodylate, pH 7.5, 300 mM KCl, 5 mM MgCl₂). After incubation at 37° C. for 20 min, 100 μM of aminoglycosides were added to the RNA mixture and incubation was continue for 15 min. Then, RNA 1-311 was added as a competitor and sample were collected at 2, 5, 10, 15, 20, 30, and 60 min and analyzed on 0.8% agarose gels (45 mM Tris borate pH 8.3, 01 mM MgCl₂) at 4° C. Gels were fixed in TCA 10%, dried, and analyzed using a BAS 2000 Bio-Imager (Fuji).

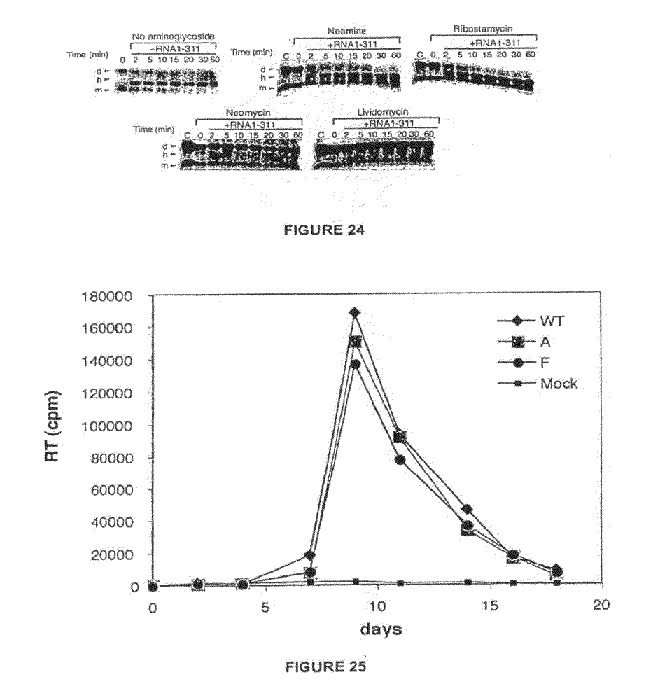

Results: Preliminary studies on a short RNA strongly suggested that aminoglycosides increase the thermal stability of the DIS kissing-loop complex (Ennifar et al., J. Biol. Chem. 2003, 278:2723-2730). Here, the effects of aminoglycosides on the dynamics of the kissing-loop complex at 37° C., in the context of RNA1-615 were studied (FIG. 24).

The kissing-loop complex of radiolabeled RNA1-615 was formed, and then aminoglycosides at a 100 _{A}M concentration were added. Next, a fivefold excess of unlabeled RNA1-311, encompassing nucleotides 1 to 311 of HIV-1 MAL genomic RNA, that also contains the subtype A DIS and is thus able to form a heterodimer with RNA1-615, was added (FIG. 24). Due to the dynamic nature of the kissing-loop complex, in the absence of aminoglycoside, greater than half of the radioactive material was shifted in the heterodimer within two minutes, and the RNA1-615 homodimer almost completely disappeared upon prolonged incubation. Similar results were obtained in the presence of 100 μM neamine and ribostamycin (FIG. 24). However, only minimal amounts of heterodimer were formed after one hour when 100 μM neomycin, paromomycin or lividomycin were present in the reaction, and the amount of RNA1-615 homodimer remained almost constant (FIG. 24). This observation demonstrates that the 4,5-disubstituted DOS that efficiently bound to the DIS dramatically reduced dissociation of the kissing-loop complex. This observation is in keeping with the crystal structures showing that 4,5-disubstituted DOS form a bridge between the two RNA molecules (FIG. 18).

### EXAMPLE 7

### Binding of Aminoglycoside Antibiotics to the DIS in Infected Cells and Virions

The ability of aminoglycosides to target the DIS in the context of the complete HIV-1 genomic RNA, in infected cells or in viral particles, has been investigated using a method recently developed by us through monitoring the DMS-induced modifications of HIV-1 genomic RNA in cells and in viral particles (Goldschmidt et al., J. Biol. Chem. 2004, 279:35923-35931; Paillart et al., J. Biol. Chem. 2004, 279:48397-48403). For the first time, we were able to detect binding to the DIS ex vivo.

Paromomycin, neomycin and lividomycin did not protect A₂₈₀ against DMS methylation of subtype B DIS in infected cells and in viral particles. However, they reduced alkylation of the subtype A and subtype F DIS more than two-fold. Subtype A and subtype F DIS were protected to the same extend in cells, while protection of subtype F DIS was systematically more efficient in virions.

These experiments demonstrate that the DIS is accessible in vivo, a key result and a prerequisite for a drug target.

Materials and Methods: To detect the footprint of aminoglycosides on the DIS of the genomic RNA, five millions CEMx174 cells were infected with equivalent amount of wild-type and mutant viruses as determined by RT activity. One hour after infection, cells were diluted in 20 ml RPMI 1640 (supplemented with 10% heat-inactivated fetal calf serum) and cultured in the absence or in the presence of 3 mM aminoglycosides.

At 72 h after infection, CEMx174 cells were washed twice with phosphate-buffered saline (PBS 1×) and suspended in 30 μl of PBS 1×. Progeny viruses were collected and purified as described (Goldschmidt et al., J. Biol. Chem. 2004, 279:35923-35931). Cells and viruses were treated with 3 μl of DMS for 4 and 8 min at 37° C. Reaction was stopped by adding 1 ml of TriReagent (Molecular Research Center), and RNA was extracted as described by the supplier. Modified bases were detected by primer extension as described (Paillart et al., J. Biol. Chem. 2004, 279:48397-48403).

Results: The next step was to test whether 4,5-disubstituted DOS are able to target the DIS in the context of the complete HIV-1 genomic RNA, in infected cells or in viral particles. It is possible to monitor modification of the HIV-1 genomic RNA by DMS in cells and in viral particles (Goldschmidt et al., J. Biol. Chem. 2004, 279:35923-35931; Paillart et al., J. Biol. Chem. 2004, 279:48397-48403). This technique was used to detect binding of 4,5-disubstituted DOS to the DIS ex vivo and appears to be the first example of footprinting of a small RNA ligand in cells or virions.

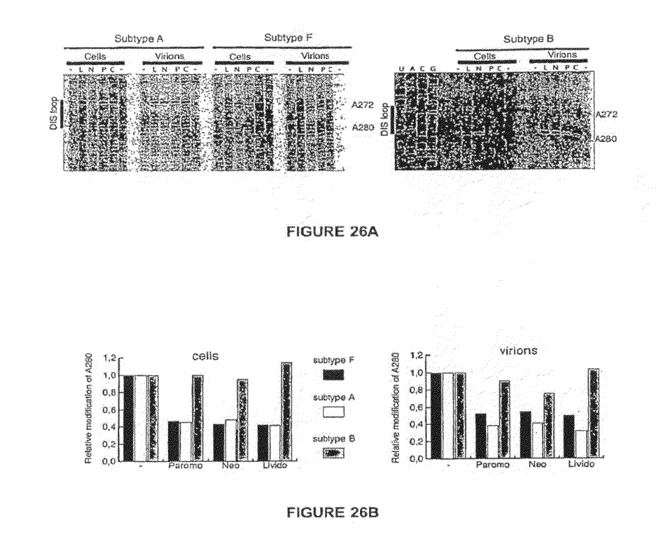

As all generally used HIV-1 laboratory strains are subtype B strains, subtype A or subtype F DIS loop sequences were substituted for the original subtype B DIS loop in the pNL4.3 infectious molecular clone. These substitutions did not affect replication of the mutant viruses (FIG. 25). As previously observed in vitro, in the absence of aminoglycoside, A₂₈₀ was more reactive in cells and virions than A₂₇₂ (FIG. 26A). Adding neomycin, paromomycin, or livodomycin to the culture medium did not completely protect A₂₈₀ from methylation by DMS, but the intensity of the band decreased, reflecting partial protection. To quantify this protection, modification of A₂₃₀ relative to A₂₇₂ was normalized, thus correcting the intensity of the bands for variations in the amount of material used in the primer extension assay. The influence of aminoglycosides on the relative modification of Aₗₛₒ is shown in FIG. 26B. Paromomycin, neomycin and lividomycin did not protect A₂₈₀ against methylation of subtype B DIS in infected cells and in viral particles. However, these three 4,5-disubstituted DOS reduced alkylation of the subtype A and subtype F DIS more than two-fold. Subtype A and subtype F DIS were protected to the same extent in cells, while protection of subtype F DIS was systematically more efficient in virions (FIG. 26B). The origin of this difference is unknown. Notably, paromomycin, neomycin and lividomycin provided similar levels of protection.

### EXAMPLE 8

### Solution Studies: Stabilization of the Loop-Loop Interaction

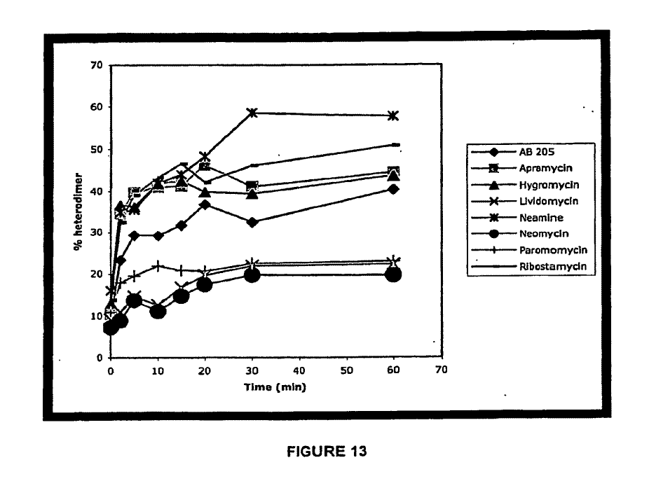

The aminoglycosides have also been investigated in solution. Their ability to stabilize the loop-loop interaction was investigated through competition experiments. Preformed RNA homodimers (1-615/1-615) were placed in the presence of the same but shorter RNA (1-311), and the kinetic of their exchanges was followed in the presence, or not, of aminoglycosides (FIG. 13).

As expected, neomycin, paromomycin and lividomycin almost totally abolish the exchange, showing a strong stabilization of the DIS dimer. Dimeric neamine derivatives also showed a strong but less efficient stabilization (FIG. 13).

It is to be understood that the invention is not limited in its application to the details of construction and parts as described hereinabove. The invention is capable of other embodiments and of being practiced in various ways. It is also understood that the phraseology or terminology used herein is for the purpose of description and not limitation. Hence, although the present invention has been described hereinabove by way of illustrative embodiments thereof, it can be modified, without departing from the spirit, scope and nature of the subject invention as defined in the appended claims.

We claim :

1. A compound having the following formula: 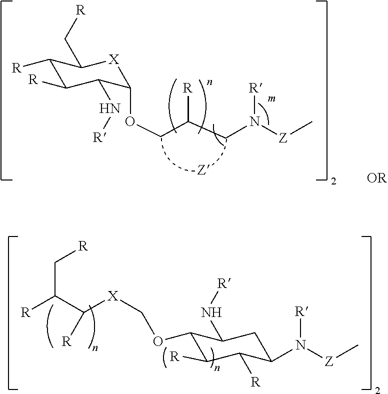
wherein X is a carbon, an oxygen, nitrogen or sulfur atom; wherein Z and Z′ are selected from the group consisting of an alkyl chain optionally containing amine or hydroxyl groups; and wherein R and R′ can be the same or different and are independently selected from the group consisting of amine or hydroxyl groups. “n, m” is a number between 1 and 5.

2. A compound selected from the group consisting of: 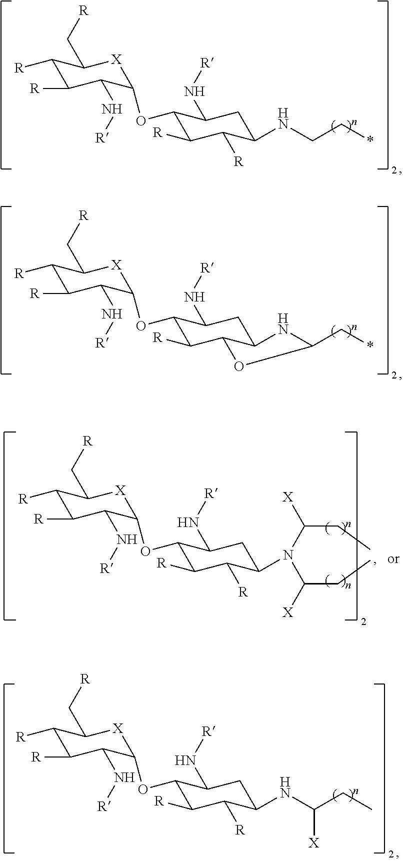
wherein R is selected from an amine or a hydroxyl group or an alkyl chain containing a hydroxyl or amine, R′ is selected from a hydrogen, oxygen or nitrogen atom or a straight or branched, saturated or unsaturated, alkyl group of between 1 and 10 carbons, X is selected from an oxygen atom, hydroxyl, nitrogen atom, amine, sulfur atom or a carbon atom and n is 1 to 15, or its pharmaceutically acceptable salt or ester.

3. A compound of claim 1, further comprising a pharmaceutical carrier, diluent, or excipient, or a prodrug thereof.

4. A compound of claim 1, wherein the compound further comprises an additive selected from an anti-oxidant, a buffer, a bacteriostat, a liquid carrier, a solute, a suspending agent, a thickening agent, a flavoring agent, a gelatin, a glycerin, a binder, a lubricant, an inert diluent, a preservative, a surface active agent, a dispersing agent, a biodegradable polymer, or any combination thereof.

5. A compound, or its pharmaceutically acceptable salt or ester, selected from the group consisting of:

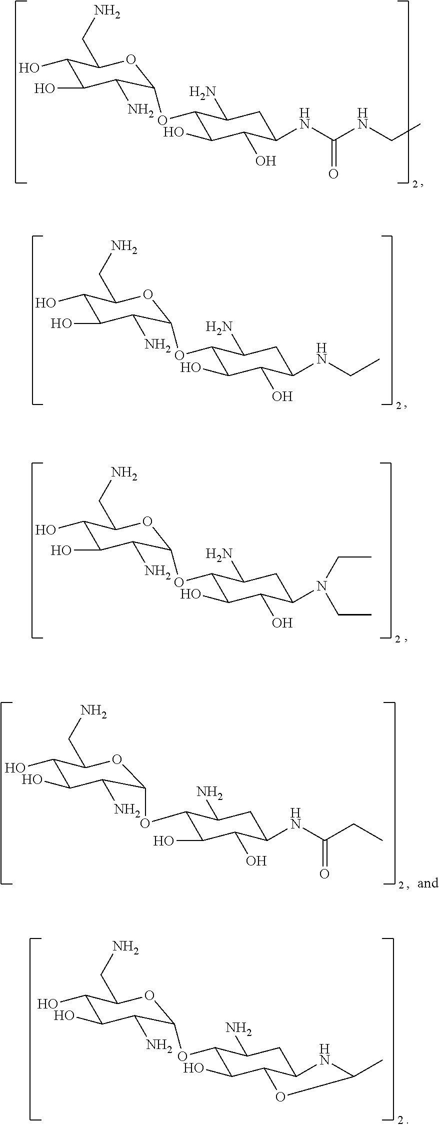

6. A compound of claim 5, further comprising a pharmaceutical carrier, diluent, or excipient, or a prodrug thereof.

7. A compound of claim 5, wherein the compound further comprises an additive selected from an anti-oxidant, a buffer, a bacteriostat, a liquid carrier, a solute, a suspending agent, a thickening agent, a flavoring agent, a gelatin, a glycerin, a binder, a lubricant, an inert diluent, a preservative, a surface active agent, a dispersing agent, a biodegradable polymer, or any combination thereof.

8. A compound comprising: 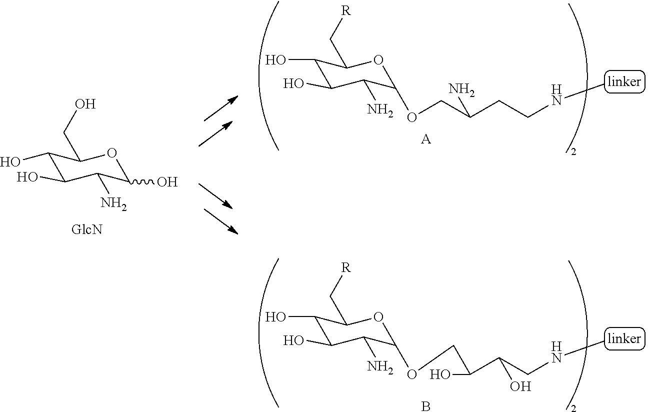
wherein R selected from an amine or a hydroxyl group or chain containing such groups, or its pharmaceutically acceptable salt or ester.

9. A compound of claim 8, further comprising a pharmaceutical carrier, diluent, or excipient, or a prodrug thereof.

10. A compound of claim 8, wherein the compound further comprises an additive selected from an anti-oxidant, a buffer, a bacteriostat, a liquid carrier, a solute, a suspending agent, a thickening agent, a flavoring agent, a gelatin, a glycerin, a binder, a lubricant, an inert diluent, a preservative, a surface active agent, a dispersing agent, a biodegradable polymer, or any combination thereof.

11. A compound comprising: 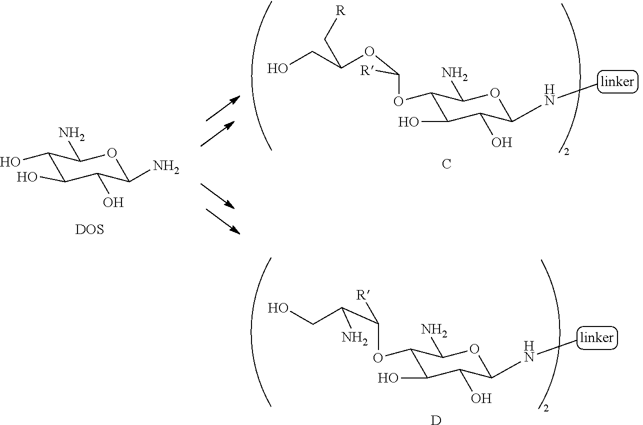
wherein R is selected from an amine or a hydroxyl group or chain containing such groups, R′ is selected from a hydrogen, oxygen or nitrogen atom or a straight or branched, saturated or unsaturated, alkyl group of between 1 and 10 carbons, or its pharmaceutically acceptable salt or ester.

12. A compound of claim 11, further comprising a pharmaceutical carrier, diluent, or excipient, or a prodrug thereof.

13. A compound of claim 11, wherein the compound further comprises an additive selected from an anti-oxidant, a buffer, a bacteriostat, a liquid carrier, a solute, a suspending agent, a thickening agent, a flavoring agent, a gelatin, a glycerin, a binder, a lubricant, an inert diluent, a preservative, a surface active agent, a dispersing agent, a biodegradable polymer, or any combination thereof.

14. A method of treating a human or animal infected with an HIV-1 virus comprising administering to the human or an animal an effective amount of the compound of claim 2.

15. The method of claim 14, wherein said compound is effective in the inhibition of HIV-1 RNA replication.

16. The method of claim 14, wherein said compound selectively targets the kissing loop complex or the corresponding duplex of genomic RNA.

17. The method of claim 14, wherein the administration of the compound is oral, parenteral, transdermal, topical, intravenous, subcutaneous, intramuscular, intradermal, ophthalmic, epidural, intratracheal, sublingual, buccal, rectal, vaginal, nasal or inhalation.

18. The method of claim 14, wherein the compound is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

19. A method of treating a human or animal infected with an HIV-1 virus comprising administering to the human or an animal an effective amount of:
a compound selected from the group consisting of: 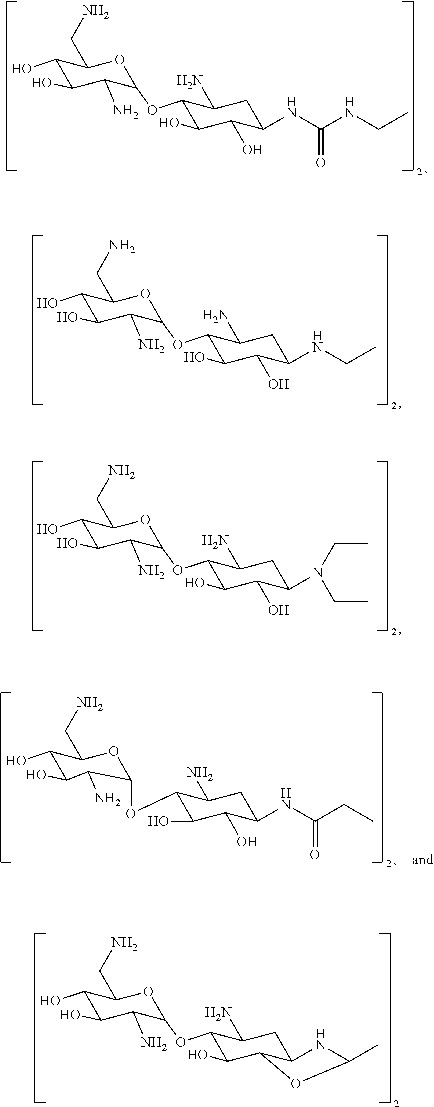
or its pharmaceutically acceptable salt or ester.

20. The method of claim 19, wherein said compound is effective in the inhibition of HIV-1 RNA replication.

21. The method of claim 19, wherein said compound selectively targets the kissing loop complex or the corresponding duplex of genomic RNA.

22. The method of claim 19, wherein the administration of the compound is oral, parenteral, transdermal, topical, intravenous, subcutaneous, intramuscular, intradermal, ophthalmic, epidural, intratracheal, sublingual, buccal, rectal, vaginal, nasal or inhalation.

23. The method of claim 19, wherein the compound is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

24. A method of treating a human or animal infected with an HIV-1 virus comprising administering to the human or an animal an effective amount of a compound comprising: 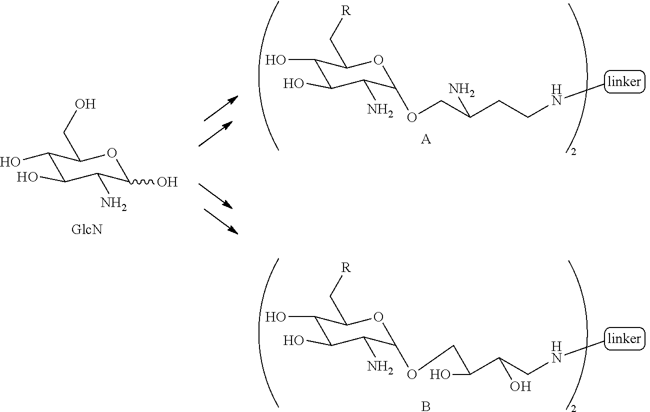
wherein R selected from an amine or a hydroxyl group or chain containing such groups, or its pharmaceutically acceptable salt or ester.

25. The method of claim 24, wherein said compound is effective in the inhibition of HIV-1 RNA replication.

26. The method of claim 24, wherein said compound selectively targets the kissing loop complex or the corresponding duplex of genomic RNA.

27. The method of claim 24, wherein the administration of the compound is oral, parenteral, transdermal, topical, intravenous, subcutaneous, intramuscular, intradermal, ophthalmic, epidural, intratracheal, sublingual, buccal, rectal, vaginal, nasal or inhalation.

28. The method of claim 24, wherein the compound is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

28. A method of treating a human or animal infected with an HIV-1 virus comprising administering to the human or an animal an effective amount of a compound comprising: 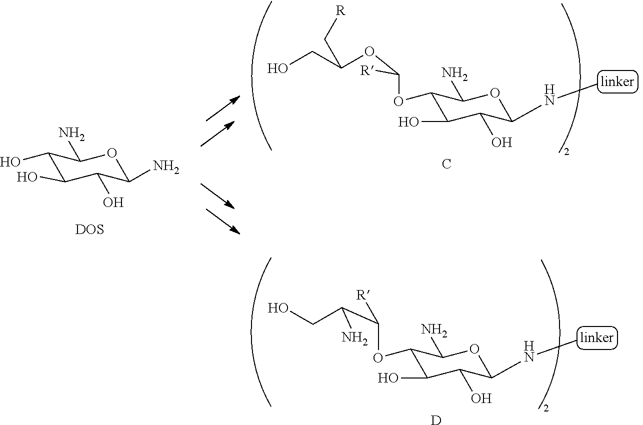
wherein R is selected from an amine or a hydroxyl group or chain containing such groups, R′ is selected from a hydrogen, oxygen or nitrogen atom or a straight or branched, saturated or unsaturated, alkyl group of between 1 and 10 carbons, or its pharmaceutically acceptable salt or ester.

29. The method of claim 28, wherein said compound is effective in the inhibition of HIV-1 RNA replication.

30. The method of claim 28, wherein said compound selectively targets the kissing loop complex or the corresponding duplex of genomic RNA.

31. The method of claim 28, wherein the administration of the compound is oral, parenteral, transdermal, topical, intravenous, subcutaneous, intramuscular, intradermal, ophthalmic, epidural, intratracheal, sublingual, buccal, rectal, vaginal, nasal or inhalation.

32. The method of claim 28, wherein the compound is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.
